SEQUENCE LISTING (48 sequences)

SEQ ID NO: 1 - DNA, 6951 bp, Sorghum bicolor
  misc_feature (2573)..(2573): polymorphic site
catgcatgccccggccgcgatctccctgtatcatgcatgttggagcagaagcacatatttttgctagccggatactgtattatttcacatcttcaagtgttatttattgcatatattggagcaccagctaacttggagaccatcaatatatccgggtgtgtgatcactgcatcgcggatatatatacatatagctccatagccgtgcagtaagtgctttattcaatatcgcatgtgaaatatactatgatgtgcgaacttatatatgctaagtgctttcaacacacagctagcgctcgatcatgttgaggtgctatatttgcatggacatggctagctttggtttaagttgtcagttctcctgctaacttgctctcaggcatccgcaatttaaatttgtgtattaagcttcatcaatcatatttttgcaaatttgcattgggatgctaggttagagataagttgatggttgtgaaaactcaatagtaatatacactgattcaaatcggtagatagctacactagagaggaaaaggtctgcaggcgatataaagatagttaataatttggctcaaatttgtagatccacatttttttgacatttgtagatccacattgaaaattagtactccctccagtgtgaaaatagttgatattttggacaatgattcattctctaaaacatattttctttgactactattttctatcagaatacaaattaatatcctaaccatttacttaagacaagtgtacatatgatcatcaagtttctaaattaaatatgttaaaaactcttttgttaccaaagttttaaaatattaactgaaatattgtccaaaatgtcaagtatttgcaaaccaaagaaagaaagttttttgtcgtacaaagtccgacggaaacaaattttatacgaaaaggccattttaatttactaccatgaaaatccacttggtccactaggcttagtttactactacaaaatgttttgaattgatccaatcgaacccaaacatagtcttttttttgtttctaataacatctataaaaagagtttttaagttcagttgttctaggccaatgctagaaatggtgccctaacatagtcacaggtttagcatctaacactaaattagtccatagagatacagaattgcatggaagtaattacaaaaaaatcttggtctatttttctaatatattacatgatagtgcaagttttattactcaaaagtcaacttgtatgcacagaaattaaaaaaaaagaaagatctcatttgggaccagattggatcaattgaaatctttttttcttggaaagtttaaatttactctattttttcaagaggttaagtggacaaattaaagttgattgttcgggggagtaaaatggacttcttccatgtacatgaaactgtagctcttcatgagttcggttcggtctatagttacatatatatagttgcattgataaaattctgtttgaagttattatcttgatccacaaatctggactacaacaaattttgtattatatttctaacttgcaatgtcaagttggacttctttctgtcgtggttcattcaagaacccctagatgacatggaatgatataaaactaagaatgaaacatggaataataatatatgtgatatattactaattaactgaatgattgacatgatgtatttaattaagtccaagttggatgtaataagggcagctccggcagtgttaaaacagctagctcatagaataagagagaatgatatgttagctcttcatgaagagctaagctcatacattttcctatggttcatgtgtctatgcttacacattttttctatactagcatgatgttatgttactccatttttatatttttttaatgtttatataaatgagctagcaacttaattaaccctatgattgagagtgccctaaaaacaatgtacaggtagttttctagtagtattggattgaagggtaaatatatatgcttgtatatctatgtggcctatagctaacatggagtaaaattgctctctctctctctctctctctcttgataatcaggtcgaatggaccgggagctgtggccttctgggctaagggtcctggttattgataacaattcttcatatttgtcagttatggaagaactacttatcaagtgcagctacaaaggtaattcattagagacgatattatatgtgtatccgaaaacatgatggataaagctttattaccattaacgcatattattattatatttttaaaacccaactgcatgtaagaccatgcggtctccaatcgtacatctcctagttatagaatatttatacaatatacaagagataatgttaactggtcaacgatattctctgtatgcttgaactattcatacagaaacttattaggtcaacgacgcgttcaatgtaaaatgattctttcagttcagtacaacacatattcgtattatactttctgacatagtttagtccccaatcacatccagttacgtcatataaggacgtcagggaagcaatgtccttcatctatggaaacatacagattgttgatctcataattagcgatgtgtgctttccaactgaagacagtttacttattctgcaagaagttaccacaaagtttgacatccccaccgtgagtaagtttatctagctctctatcctttactctttccgtcttaaaatatcccgacaatataattttaggatcaaataaagaagtctggcaaaaaaaaactaataacccgctcataactagttagtttggttattagcttgctcccgcctcttactaattagttagtttggtgttagcttgcttaatttctagcaacgccgtggcagtttggaaaaaaaaagaacatgcatgtcactgtttttgctacatgatgtttaaaagtgaaggaccagaaacaatggccagaaggggtgggatgaatagggcctcaaaaattttctgataaaaatgacgtggaggcctatatcttgataccacgaatctcacaaatacaactagccaaaaacatgtaatcacaagtccttataaagtaggttcagaagatacgtaagcaaacaaagatcaaaacgagcaaacttggaaaccaatagcaaaaattgtttcagccagtttttttagaacatagctctgagaagaagaaaagtcaaaacctgatcgaatgacctcaaacatcatgaaattttatggagaactttgtaacgccgtagaggatatctcaacaaaaaatcagctcaaacagagcaatttgaaaaaaccaacctagactggtttgtgaaaccagcagaatcctccctaagaaaaatttaaaataatttcaaataagctctgaaaattcagaaacttagcacataccttgactaacccacttagaatctatccccatgagatcaactacaaaagatgaacacttgaacataaatttcaaattgtggtcttgtgtcaagaatcccccaaatcatgatttctaggtgctcgtgggtaatcagtagtcactttgcaaggtccctcagcacctaaagatcaatcacaaacaacccaaaaccttccgataaaagatgtctaagaaaatcaccaaaagagacttaaaaagtcaagttttgggaaggaaacttacaagatgagagctgggcttgaagccatggtttgaacaagttctaagagtcccaaaacactccaaattgaggctgcaacatagccaaaccagtagattgaaaaacactaaaaatgactatgaaaatcactgaaaaaggaaaaattggaggagggattgactcgagatttcaaccaagaacttgattaaacaaccatgaaatcttctttacaagaccaggtgtatcctcctcccttcatcctcccactagagatggaaaactagagcaagaggtgcttatctctctctacttctaatctcctgcacaagaagcatttggctagtctaaaggaagaggtggctgtcttgggcagcttctatccttatttatagggctcttacataattccaagagtgctcctaggttcaggcatgaaccacttagccacaaggacaaaatggtcgaaattgatttccatacgtacattgggtgggcacgagtccttcatgaagtcgctttgcctcaacgcaagctcttcgatgacgctagagatcctcccccaaattttgaggcccaaatttggcaaacttgcttcgcgtggttttcaggcccaaactaccaaacccacttggaattgtgtagccgctacaccttcttcacgatgtcattatgtgtcgtctttgaacgactaatcacctagtgcccgcacgtccgctttgactttgtcttatcgccgtcttgacttcagtcaacaccgtctcgacttcagtcaacaccttctccatcaccatcgcatgtacacttgcttgtggatgtgtctaggtgctagccatccacggtctgtcctctagccctaggtctctcagtccaagcctttgtgtctacccttcagctagtgctcctggtctatcacacacagactcctcgcttgaccttctccattgccatcgatcgtctcatgctcaacacctgcatatcacattaccaagagacatgttgcacacacacacacacacaactctcataaaaacttaaccaaagttctgatcacctcattgacagtcactcatcactcacacatgatcacatatcaaccatgtgttcgcaaaaaaaaacatgcgtaaaaggtgtttaattaaaaggcaagcacatatgtgaggttgagcaatataattcataataccttataatttgggagaatttttaaacctttaataccttgtattttaggatagagggagtactctccattctctatagaaagtttcttaaatttgtcagattgattcattaggttttattctgcttgcatgatggttttactgtttctgattttgcttgtcacatctcccgtgactaagtttatctagatctctattacttattctcagttacacatagaaactttctcaaatatttatcagatagatgcattagttttatttgattttgtatgacggttttttttattttgcttgccatgacctcttgatggcccacgggtctatgatttcctaagtagtacaactacttgcatctatctgatatgggttagctattagctattccagttgagtttagaagtatgtacctttctcatttatagccatgttaaccagtaatgttttcaaacggtgatacgagctatgtgttcactaagcttttcatattatatgtccatgttaaccagtaatgtcttccaacggtgatgcgagcatagttatgaagtacatcaccagtggggcttccgatttcctgataaagcctgtgagaattgaagtgctgaagaacatatggcagcatgtgttccggaagcagctgatcggggagaacagaagctgcagcaacagtgctcaacacctcgatcaggtttcatatccaccaaccatagctcctgcttctacatgtgccacaagaaccacaggaataatcactgaagcagccacggcgacactggagagcgcgacaagagagacgactaacgggacggtcacagatatacaggatctgaggaagtcaaggctcagctggaccacacagctgcaccgccaattcattgctgctgtgaattccctgggggaaagtgagtagtgatcaaccacatttccttagctttaagcatgttcgtatatatatattaataaagtacgtgtttaattttctttgtgtttaattatatatattggttatatatacaatattggtttacagaggcagttccaaagaagatactggagacaatgaaggttaaacatttgacaagagaacaagttgcaagccacttgcaggtaattaattagtatattctgatatatacttatgttcttgtgcaaggcctactggcatgcatgccagacgaatatgcatgtctcctatttaggttaaacattggacaagagaacaagttgcatgatagatatctccatcattaaaagtccatgattatgttaatgtttcttttttttgcaaaggcatgattattaatgttgacttaaactaatatacaaaaatacaattattttgcatgcatgtgctaataacctagctcaaataattcgttcaagaactgcatatgatggatcatttcattttgtaatactcctgtaatataattatgttgtttgtggatatactgatgaaccataaaaattctacttcaaaaaactgatgcagaaatacaggcttcacctaaggaaattgaatcaaacattgcacaaggatgacacaccttcaccatcaagccatcccaatgaatcaaacattctccgaaccgaattcaatagttctttgaattcaacgtattttgatcaagatggatgcttggagatcacagagtactctttgcccaaggatgacatctcaagtggttcagactgtatgctgggagaacgaaacaactactcacctcaaggcttccaggatttcagatgggattcagagaaacagggatctgaaacgacatatttatggaatttcgaggcagagtgattctatcactataaacatgcatcataccaccaaacataactaggtaatgatgtgcacttatatgccatgtcctgaataatgacagcttatatatgtctagagtattacgtcctgtaggctgtactgaacctcattgtgtatgtgttgcctacttgagaataatactggagcgaacaagaactactttactactatatagccagacctacttaatttgtaattatgtaattatgcacttataattccctacaggaaacaactaagatatgtatgtttctggcttttctataagcatgtaataggtattactgtttcagttgttt

SEQ ID NO: 2 - DNA, 1580 bp, Sorghum bicolor
  misc_feature (375)..(375): polymorphic site
catgcatgccccggccgcgatctccctgtatcatgcatgttggagcagaagcacatatttttgctagccggatactgtattatttcacatcttcaagtgttatttattgcatatattggagcaccagctaacttggagaccatcaatatatccgggtgtgtgatcactgcatcgcggatatatatacatatagctccatagccgtgcagtcgaatggaccgggagctgtggccttctgggctaagggtcctggttattgataacaattcttcatatttgtcagttatggaagaactacttatcaagtgcagctacaaagttacgtcatataaggacgtcagggaagcaatgtccttcatctatggaaacatacagattgttgatctcataattagcgatgtgtgctttccaactgaagacagtttacttattctgcaagaagttaccacaaagtttgacatccccaccgtgataatgtcttccaacggtgatgcgagcatagttatgaagtacatcaccagtggggcttccgatttcctgataaagcctgtgagaattgaagtgctgaagaacatatggcagcatgtgttccggaagcagctgatcggggagaacagaagctgcagcaacagtgctcaacacctcgatcaggtttcatatccaccaaccatagctcctgcttctacatgtgccacaagaaccacaggaataatcactgaagcagccacggcgacactggagagcgcgacaagagagacgactaacgggacggtcacagatatacaggatctgaggaagtcaaggctcagctggaccacacagctgcaccgccaattcattgctgctgtgaattccctgggggaaaaggcagttccaaagaagatactggagacaatgaaggttaaacatttgacaagagaacaagttgcaagccacttgcagaaatacaggcttcacctaaggaaattgaatcaaacattgcacaaggatgacacaccttcaccatcaagccatcccaatgaatcaaacattctccgaaccgaattcaatagttctttgaattcaacgtattttgatcaagatggatgcttggagatcacagagtactctttgcccaaggatgacatctcaagtggttcagactgtatgctgggagaacgaaacaactactcacctcaaggcttccaggatttcagatgggattcagagaaacagggatctgaaacgacatatttatggaatttcgaggcagagtgattctatcactataaacatgcatcataccaccaaacataactaggtaatgatgtgcacttatatgccatgtcctgaataatgacagcttatatatgtctagagtattacgtcctgtaggctgtactgaacctcattgtgtatgtgttgcctacttgagaataatactggagcgaacaagaactactttactactatatagccagacctacttaatttgtaattatgtaattatgcacttataattccctacaggaaacaactaagatatgtatgtttctggcttttctataagcatgtaataggtattactgtttcagttgttt

SEQ ID NO: 3 - DNA, 1044 bp, Sorghum bicolor
  misc_feature (162)..(162): polymorphic site
atggaccgggagctgtggccttctgggctaagggtcctggttattgataacaattcttcatatttgtcagttatggaagaactacttatcaagtgcagctacaaagttacgtcatataaggacgtcagggaagcaatgtccttcatctatggaaacatacagattgttgatctcataattagcgatgtgtgctttccaactgaagacagtttacttattctgcaagaagttaccacaaagtttgacatccccaccgtgataatgtcttccaacggtgatgcgagcatagttatgaagtacatcaccagtggggcttccgatttcctgataaagcctgtgagaattgaagtgctgaagaacatatggcagcatgtgttccggaagcagctgatcggggagaacagaagctgcagcaacagtgctcaacacctcgatcaggtttcatatccaccaaccatagctcctgcttctacatgtgccacaagaaccacaggaataatcactgaagcagccacggcgacactggagagcgcgacaagagagacgactaacgggacggtcacagatatacaggatctgaggaagtcaaggctcagctggaccacacagctgcaccgccaattcattgctgctgtgaattccctgggggaaaaggcagttccaaagaagatactggagacaatgaaggttaaacatttgacaagagaacaagttgcaagccacttgcagaaatacaggcttcacctaaggaaattgaatcaaacattgcacaaggatgacacaccttcaccatcaagccatcccaatgaatcaaacattctccgaaccgaattcaatagttctttgaattcaacgtattttgatcaagatggatgcttggagatcacagagtactctttgcccaaggatgacatctcaagtggttcagactgtatgctgggagaacgaaacaactactcacctcaaggcttccaggatttcagatgggattcagagaaacagggatctgaaacgacatatttatggaatttcgaggcagagtga

SEQ ID NO: 4 - protein, 347 aa, Sorghum bicolor
  MISC_FEATURE (189)..(189): polymorphic site
MDRELWPSGLRVLVIDNNSSYLSVMEELLIKCSYKVTSYKDVREAMSFIYGNIQIVDLIISDVCFPTEDSLLILQEVTTKFDIPTVIMSSNGDASIVMKYITSGASDFLIKPVRIEVLKNIWQHVFRKQLIGENRSCSNSAQHLDQVSYPPTIAPASTCATRTTGIITEAATATLESATRETTNGTVTDIQDLRKSRLSWTTQLHRQFIAAVNSLGEKAVPKKILETMKVKHLTREQVASHLQKYRLHLRKLNQTLHKDDTPSPSSHPNESNILRTEFNSSLNSTYFDQDGCLEITEYSLPKDDISSGSDCMLGERNNYSPQGFQDFRWDSEKQGSETTYLWNFEAE

SEQ ID NO: 5 - DNA, 2157 bp, Sorghum bicolor
  misc_feature (605)..(605): polymorphic site
gcgttgttcacttcacttgacttccccttagcttccacccacaaactcatacgtactcgctccgacagtccctcactgcacacaagacacatagcaggcataggataggagccagccatgagaggtagaaggacaggagaagacatatacacccacatatagtgaagggaacacagtagtcacccagtcggtcctgtggcagctagcttgctacaacaggctgctggtctggtctctgtgcgtggtgcaaccagtgcaagtgacaaattaaccttgccgagatccgttcatgaattataacttcagctccaacgctctcgacgaggaggaggtcgctggaagaggcggggaaggagggagctgcgccgcagcaccagcatgggccaggccctgcgacgggtgccgggcggcgcccagcgtggtgtactgccacgccgacgcggcgtacctgtgcgcgtcgtgcgacgtgcgggtgcacgccgccaaccgcgttgcgtcgcgccacgagcgcgtgcgcgtgtgcgaggcctgcgagcgcgcgccagcagtgctggcgtgccgcgccgacgccgccgcgctctgcgtcgtctgcgacgcgcaggtccactccgcgaacccgctggccgggaggcaccagcgcgtgcccgtgctgccgctccccgtcgcggccatcccggctgcttccgtgctcgccgaggctgcggccaccgccgtggccgtgggtgacaagcaggaagaggaggtggactcgtggctgctgctcaccaacaccaaggatccagtttcagacaacaacaactgcaactgcagcagcagcagcaacaacaacatcagcagcagcaacaccagcaccttctacgcggatgttgatgagtactttgatctcgtgggctacaattcctactgtgacaaccacatcaacagcaacccaaagcagtacgggatgcaagaacgacagcaacagcagcagctgctgctgcaaaaggaatttggagacaaggagggaagcgagcacgttgtgcctgcttcacaggtcgcgatggcaaatgagcagcagcagagtggttatggagttattggggtagagcaggctgcctccatgactgccgcggtcagtgcttacacagattccatcactaacagcgtgagtccatctattactactattactatcactatatatatatatgtgtgtgtttgttttgaggtttcaatgttactttactataatataagagggtaagtagtccaaatttatccctaccatgagcagaattaacgtccataaattaaacacatgctatctactacatatcattgcatgcagggtcgtctatgacacctgcaatccccttatgattcgcatatttcagtgaccatttaccgattccatctcagatatctttctcatcatcaatggaggtgggtatagtcccagacaacatggcaacgacgacagacatgccaaactccggcatcctgctgacacctgctgaggccatcagcctcttctcgtcaggttcttcgcttcagatgccactccacttgacctccatggacagagaggccagggtcctcaggtacaaggagaagaagaagagcagaaagttcgcgaagaccatacgatatgcgacgaggaagacatatgcagaagcaaggccgaggatcaagggccgcttcgccaagagatcttctgatatggaaatcgaagtggaccagatgttctcatctgcagctctgtcgtctgatggtagctacggtacggttctatggttctgaagggactttcgtgagacattattaccatatatatatatatgtaataatagaacatgtgttgaccatattgataaggtcgagtgtacaagtagttctaggaagccgatgctatgagtggtattgtgtttgtttgaaaacttaaatgaacaattaccttagcatttgagttttcctttgtaattgcttatagaggtctacatatactgttagtacccgctcagtcactcttaacatttttatgtgaacagagagcaaaaaaaaaataaagtggaattgggaattagtgtgcaaatgaatctttgttgccacagatcaattagcaacagaattattgtcaaaaaaaagtttagcaacataatatatatagcagtaatgcta

SEQ ID NO: 6 - DNA, 1903 bp, Sorghum bicolor
  misc_feature (605)..(605)
gcgttgttcacttcacttgacttccccttagcttccacccacaaactcatacgtactcgctccgacagtccctcactgcacacaagacacatagcaggcataggataggagccagccatgagaggtagaaggacaggagaagacatatacacccacatatagtgaagggaacacagtagtcacccagtcggtcctgtggcagctagcttgctacaacaggctgctggtctggtctctgtgcgtggtgcaaccagtgcaagtgacaaattaaccttgccgagatccgttcatgaattataacttcagctccaacgctctcgacgaggaggaggtcgctggaagaggcggggaaggagggagctgcgccgcagcaccagcatgggccaggccctgcgacgggtgccgggcggcgcccagcgtggtgtactgccacgccgacgcggcgtacctgtgcgcgtcgtgcgacgtgcgggtgcacgccgccaaccgcgttgcgtcgcgccacgagcgcgtgcgcgtgtgcgaggcctgcgagcgcgcgccagcagtgctggcgtgccgcgccgacgccgccgcgctctgcgtcgtctgcgacgcgcaggtccactccgcgaacccgctggccgggaggcaccagcgcgtgcccgtgctgccgctccccgtcgcggccatcccggctgcttccgtgctcgccgaggctgcggccaccgccgtggccgtgggtgacaagcaggaagaggaggtggactcgtggctgctgctcaccaacaccaaggatccagtttcagacaacaacaactgcaactgcagcagcagcagcaacaacaacatcagcagcagcaacaccagcaccttctacgcggatgttgatgagtactttgatctcgtgggctacaattcctactgtgacaaccacatcaacagcaacccaaagcagtacgggatgcaagaacgacagcaacagcagcagctgctgctgcaaaaggaatttggagacaaggagggaagcgagcacgttgtgcctgcttcacaggtcgcgatggcaaatgagcagcagcagagtggttatggagttattggggtagagcaggctgcctccatgactgccgcggtcagtgcttacacagattccatcactaacagcatatctttctcatcatcaatggaggtgggtatagtcccagacaacatggcaacgacgacagacatgccaaactccggcatcctgctgacacctgctgaggccatcagcctcttctcgtcaggttcttcgcttcagatgccactccacttgacctccatggacagagaggccagggtcctcaggtacaaggagaagaagaagagcagaaagttcgcgaagaccatacgatatgcgacgaggaagacatatgcagaagcaaggccgaggatcaagggccgcttcgccaagagatcttctgatatggaaatcgaagtggaccagatgttctcatctgcagctctgtcgtctgatggtagctacggtacggttctatggttctgaagggactttcgtgagacattattaccatatatatatatatgtaataatagaacatgtgttgaccatattgataaggtcgagtgtacaagtagttctaggaagccgatgctatgagtggtattgtgtttgtttgaaaacttaaatgaacaattaccttagcatttgagttttcctttgtaattgcttatagaggtctacatatactgttagtacccgctcagtcactcttaacatttttatgtgaacagagagcaaaaaaaaaataaagtggaattgggaattagtgtgcaaatgaatctttgttgccacagatcaattagcaacagaattattgtcaaaaaaaagtttagcaacataatatatatagcagtaatgcta

SEQ ID NO: 7 - DNA, 1236 bp, Sorghum bicolor
  misc_feature (316)..(316): polymorphic site
atgaattataacttcagctccaacgctctcgacgaggaggaggtcgctggaagaggcggggaaggagggagctgcgccgcagcaccagcatgggccaggccctgcgacgggtgccgggcggcgcccagcgtggtgtactgccacgccgacgcggcgtacctgtgcgcgtcgtgcgacgtgcgggtgcacgccgccaaccgcgttgcgtcgcgccacgagcgcgtgcgcgtgtgcgaggcctgcgagcgcgcgccagcagtgctggcgtgccgcgccgacgccgccgcgctctgcgtcgtctgcgacgcgcaggtccactccgcgaacccgctggccgggaggcaccagcgcgtgcccgtgctgccgctccccgtcgcggccatcccggctgcttccgtgctcgccgaggctgcggccaccgccgtggccgtgggtgacaagcaggaagaggaggtggactcgtggctgctgctcaccaacaccaaggatccagtttcagacaacaacaactgcaactgcagcagcagcagcaacaacaacatcagcagcagcaacaccagcaccttctacgcggatgttgatgagtactttgatctcgtgggctacaattcctactgtgacaaccacatcaacagcaacccaaagcagtacgggatgcaagaacgacagcaacagcagcagctgctgctgcaaaaggaatttggagacaaggagggaagcgagcacgttgtgcctgcttcacaggtcgcgatggcaaatgagcagcagcagagtggttatggagttattggggtagagcaggctgcctccatgactgccgcggtcagtgcttacacagattccatcactaacagcatatctttctcatcatcaatggaggtgggtatagtcccagacaacatggcaacgacgacagacatgccaaactccggcatcctgctgacacctgctgaggccatcagcctcttctcgtcaggttcttcgcttcagatgccactccacttgacctccatggacagagaggccagggtcctcaggtacaaggagaagaagaagagcagaaagttcgcgaagaccatacgatatgcgacgaggaagacatatgcagaagcaaggccgaggatcaagggccgcttcgccaagagatcttctgatatggaaatcgaagtggaccagatgttctcatctgcagctctgtcgtctgatggtagctacggtacggttctatggttctga

SEQ ID NO: 8 - protein, 411 aa, Sorghum bicolor
  MISC_FEATURE (106)..(106): polymorphic site
MNYNFSSNALDEEEVAGRGGEGGSCAAAPAWARPCDGCRAAPSVVYCHADAAYLCASCDVRVHAANRVASRHERVRVCEACERAPAVLACRADAAALCVVCDAQVHSANPLAGRHQRVPVLPLPVAAIPAASVLAEAAATAVAVGDKQEEEVDSWLLLTNTKDPVSDNNNCNCSSSSNNNISSSNTSTFYADVDEYFDLVGYNSYCDNHINSNPKQYGMQERQQQQQLLLQKEFGDKEGSEHVVPASQVAMANEQQQSGYGVIGVEQAASMTAAVSAYTDSITNSISFSSSMEVGIVPDNMATTTDMPNSGILLTPAEAISLFSSGSSLQMPLHLTSMDREARVLRYKEKKKSRKFAKTIRYATRKTYAEARPRIKGRFAKRSSDMEIEVDQMFSSAALSSDGSYGTVLWF

SEQ ID NO: 9 - DNA, 1044 bp, Sorghum bicolor; identical to SEQ ID NO: 3

SEQ ID NO: 10 - DNA, 1044 bp, Sorghum bicolor; identical to SEQ ID NO: 3

SEQ ID NO: 11 - DNA, 1044 bp, Sorghum bicolor
atggaccgggagctgtggccttctgggctaagggtcctggttattgataacaattcttcatatttgtcagttatggaagaactacttatcaagtgcagctacaaagttacgtcatataaggacgtcagggaagcaatgtccttcatctatggaaacatacaaattgttgatctcataattagcgatgtgtgctttccaactgaagacagtttacttattctgcaagaagttaccacaaagtttgacatccccaccgtgataatgtcttccaacggtgatgcgagcatagttatgaagtacatcaccagtggggcttccgatttcctgataaagcctgtgagaattgaagtgctgaagaacatatggcagcatgtgttccggaagcagctgatcggggagaacagaagctgcagcaacagtgctcaacacctcgatcaggtttcatatccaccaaccatagctcctgcttctacatgtgccacaagaaccacaggaataatcactgaagcagccacggcgacactggagagcgcgacaagagagacgactaacgggacggtcacaaatatacaggatctgaggaagtcaaggctcagctggaccatacagctgcaccgccaattcattgctgctgtgaattccctgggggaaaaggcagttccaaagaagatactggagacaatgaaggttaaacatttgacaagagaacaagttgcaagccacttgcagaaatacaggcttcacctaaggaaattgaatcaaacattgcacaaggatgacacaccttcaccatcaagccatcccaatgaatcaaacattctccgaaccgaattcaatagttctttgaattcaacgtattttgatcaagatggatgcttggagatcacagagtactctttgcccaaggatgacatctcaagtggttcagactgtatgctgggagaacgaaacaactactcacctcaaggcttccaggatttcagatgggattcagagaaacagggatctgaaacgacatatttatggaatttcgaggcagagtga

SEQ ID NO: 12 - DNA, 1044 bp, Sorghum bicolor
atggaccgggagctgtggccttctgggctaagggtcctggttattgataacaattcttcatatttgtcagttatggaagaactacttatcaagtgcagctacaaagttacgtcatataaggacgtcagggaagcaatgtccttcatctatggaaacatacaaattgttgatctcataattagcgatgtgtgctttccaactgaagacagtttacttattctgcaagaagttaccacaaagtttgacatccccaccgtgataatgtcttccaacggtgatgcgagcatagttatgaagtacatcaccagtggggcttccgatttcctgataaagcctgtgagaattgaagtgctgaagaacatatggcagcatgtgttccggaagcagctgatcggggagaacagaagctgcagcaacagtgctcaacacctcgatcaggtttcatatccaccaaccatagctcctgcttctacatgtgccacaagaaccacaggaataatcactgaagcagccacggcgacactggagagcgcgacaagagagacgactaacgggacggtcacagatatacaggatctgaggaagtcaaggctcagctggaccatacagctgcaccgccaattcattgctgctgtgaattccctgggggaaaaggcagttccaaagaagatactggagacaatgaaggttaaacatttgacaagagaacaagttgcaagccacttgcagaaatacaggcttcacctaaggaaattgaatcaaacattgcacaaggatgacacaccttcaccatcaagccatcccaaagaatcaaacattctccgaaccgaattcaatagttctttgaattcaacgtattttgatcaagatggatgcttggagatcacagagtactctttgcccaaggatgacatctcaagtggttcagactgtatgctgggagaacgaaacaactactcacctcaaggcttccaggatttcagatgggattcagagaaacagggatctgaaacgacatatttatggaatttcgaggcagagtga

SEQ ID NO: 13 - DNA, 1044 bp, Sorghum bicolor
atggaccgggagctgtggccttctgggctaagggtcctggttattgataacaattcttcatatttgtcagttatggaagaactacttatcaagtgcagctacaaagttacgtcatataaggacgtcagggaagcaatgtccttcatctatggaaacatacaaattgttgatctcataattagcgatgtgtgctttccaactgaagacagtttacttattctgcaagaagttaccacaaagtttgacatccccaccgtgataatgtcttccaacggtgatgcgagcatagttatgaagtacatcaccagtggggcttccgatttcctgataaagcctgtgagaattgaagtgctgaagaacatatggcagcatgtgttccggaagcagctgatcggggagaacagaagctgcagcaacagtgctcaacacctcgatcaggtttcatatccaccaaccatagctcctgcttctacatgtgccacaagaaccacaggaataatcactgaagcagccacggcgacactggagagcgcgacaagagagacgactaacgggacggtcacagatatacaggatctgaggaagtcaaggctcagctgggccatacagctgcaccgccaattcattgctgctgtgaattccctgggggaaaaggcagttccaaagaagatactggagacaatgaaggttaaacatttgacaagagaacaagttgcaagccacttgcagaaatacaggcttcacctaaggaaattgaatcaaacattgcacaaggatgacacaccttcaccatcaagccatcccaatgaatcaaacattctccgaaccgaattcaatagttctttgaattcaacgtattttgatcaagatggatgcttggagatcacagagtactctttgcccaaggatgacatctcaagtggttcagactgtatgctgggagaacgaaacaactactcacctcaaggcttccaggatttcagatgggattcagagaaacagggatctgaaacgacatatttatggaatttcgaggcagagtga

SEQ ID NO: 14 - DNA, 1044 bp, Sorghum bicolor; identical to SEQ ID NO: 13

SEQ ID NO: 15 - DNA, 1044 bp, Sorghum bicolor; identical to SEQ ID NO: 13

SEQ ID NO: 16 - protein, 347 aa, Sorghum bicolor; identical to SEQ ID NO: 4

SEQ ID NO: 17 - protein, 347 aa, Sorghum bicolor; identical to SEQ ID NO: 4

SEQ ID NO: 18 - protein, 347 aa, Sorghum bicolor
MDRELWPSGLRVLVIDNNSSYLSVMEELLIKCSYKVTSYKDVREAMSFIYGNIQIVDLIISDVCFPTEDSLLILQEVTTKFDIPTVIMSSNGDASIVMKYITSGASDFLIKPVRIEVLKNIWQHVFRKQLIGENRSCSNSAQHLDQVSYPPTIAPASTCATRTTGIITEAATATLESATRETTNGTVTNIQDLRKSRLSWTIQLHRQFIAAVNSLGEKAVPKKILETMKVKHLTREQVASHLQKYRLHLRKLNQTLHKDDTPSPSSHPNESNILRTEFNSSLNSTYFDQDGCLEITEYSLPKDDISSGSDCMLGERNNYSPQGFQDFRWDSEKQGSETTYLWNFEAE

SEQ ID NO: 19 - protein, 347 aa, Sorghum bicolor
MDRELWPSGLRVLVIDNNSSYLSVMEELLIKCSYKVTSYKDVREAMSFIYGNIQIVDLIISDVCFPTEDSLLILQEVTTKFDIPTVIMSSNGDASIVMKYITSGASDFLIKPVRIEVLKNIWQHVFRKQLIGENRSCSNSAQHLDQVSYPPTIAPASTCATRTTGIITEAATATLESATRETTNGTVTDIQDLRKSRLSWTIQLHRQFIAAVNSLGEKAVPKKILETMKVKHLTREQVASHLQKYRLHLRKLNQTLHKDDTPSPSSHPKESNILRTEFNSSLNSTYFDQDGCLEITEYSLPKDDISSGSDCMLGERNNYSPQGFQDFRWDSEKQGSETTYLWNFEAE

SEQ ID NO: 20 - protein, 347 aa, Sorghum bicolor
MDRELWPSGLRVLVIDNNSSYLSVMEELLIKCSYKVTSYKDVREAMSFIYGNIQIVDLIISDVCFPTEDSLLILQEVTTKFDIPTVIMSSNGDASIVMKYITSGASDFLIKPVRIEVLKNIWQHVFRKQLIGENRSCSNSAQHLDQVSYPPTIAPASTCATRTTGIITEAATATLESATRETTNGTVTDIQDLRKSRLSWAIQLHRQFIAAVNSLGEKAVPKKILETMKVKHLTREQVASHLQKYRLHLRKLNQTLHKDDTPSPSSHPNESNILRTEFNSSLNSTYFDQDGCLEITEYSLPKDDISSGSDCMLGERNNYSPQGFQDFRWDSEKQGSETTYLWNFEAE

SEQ ID NO: 21 - protein, 347 aa, Sorghum bicolor; identical to SEQ ID NO: 20

SEQ ID NO: 22 - protein, 347 aa, Sorghum bicolor; identical to SEQ ID NO: 20

SEQ ID NO: 23 - protein, 117 aa, Sorghum bicolor
LVMDENGVSRMVTKGLLVHLGCEVTTVSSNEECLRVVSHEHKVVFMDVCMPGVENYQIALRIHEKFTKQRHQRPLLVALSGNTDKSTKEKCMSFGLDGVLLKPVSLDNIRDVLSDLL

SEQ ID NO: 24 - protein, 114 aa, Sorghum bicolor
LVIDNNSSYLSVMEELLIKCSYKVTSYKDVREAMSFIYGNIQIVDLIISDVCFPTEDSLLILQEVTTKFDIPTVIMSSNGDASIVMKYITSGASDFLIKPVRIEVLKNIWQHVF

SEQ ID NO: 25 - protein, 113 aa, Sorghum bicolor
HIVDDEEPVRKSLAFMLTMNGFAVKMHQSAEAFLAFAPDVRNGVLVTDLRMPDMSGVELLRNLGDLKINIPSIVITGHGDVPMAVEAMKAGAVDFIEKPFEDTVIIEAIERAS

SEQ ID NO: 26 - protein, 125 aa, Sorghum bicolor
LSVDDSALMRQIMTEIINSHSDMEMVATAPDPLVARDLIKKFNPDVLTLDVEMPRMDGLDFLEKLMRLRPMPVVMVSSLTGKGSEVTLRALELGAIDFVTKPQLGIREGMLAYSEMIAEKVRTAA

SEQ ID NO: 27 - protein, 112 aa, Sorghum bicolor
LLIEDDEAIRTALELSLTRQGHRVATAASGEDGLKLLREQRPDLIVLDVMLPGIDGFEVCRRIRRTDQLPIILLTARNDDIDVVVGLESGADDYVVKPVQGRVLDARIRAVL

SEQ ID NO: 28 - protein, 114 aa, Sorghum bicolor
LIVEDECAIREMIALFLSQKYYDVIEASDFKTAINKIKENPKLILLDWMLPGRSGIQFIQYIKKQESYAAIPIIMLTAKSTEEDCIACLNAGADDYITKPFSPQILLARIEAVW

SEQ ID NO: 29 - protein, 113 aa, Sorghum bicolor
LLCEDDENLGMLLREYLQAKGYSAELYPDGEAGFKAFLKNKYDLCVFDVMMPKKDGFTLAQEVRAANAEIPIIFLTAKTLKEDILEGFKIGADDYITKPFSMEELTFRIEAIL

SEQ ID NO: 30 - protein, 115 aa, Sorghum bicolor
LAIDDSRTIRELLREALVQAGFEVHLAIDGLDGLEKLEAAKPHAVITDINMPRMDGFGFIRAVREQPQHSALPIIVLTTESAAELKAKAREAGATAWIVKPFDEAKLVSALRRVA

SEQ ID NO: 31 - protein, 111 aa, Sorghum bicolor
LVVEDDEDIGDLLEESLTRAGYEVLRAKDGKRALQLVNDSLDLVILDIMMPGISGIETCQHIRKSSNVPILFLTARSSTLDKTEGLLAGGDDYMTKPFSEEELHARVIAQL

SEQ ID NO: 32 - protein, 113 aa, Sorghum bicolor
LIAEDEALIRMDLAEMLREEGYEIVGEAGDGQEAVELAELHKPDLVIMDVKMPRRDGIDAASEIASKRIAPIVVLTAFSQRDLVERARDAGAMAYLVKPFSISDLIPAIELAV

SEQ ID NO: 33 - protein, 46 aa, Sorghum bicolor
NPRMHWTDDLDIRFIQVIEKLGESKRIKRITISHVKSHLQMYRNKK

SEQ ID NO: 34 - protein, 50 aa, Sorghum bicolor
KSRLSWTTQLHRQFIAAVNSLGEKKKIETMKVKHTREQVASHLQKYRLHL

SEQ ID NO: 35 - protein, 50 aa, Sorghum bicolor
KRRVVWDEELHQNFLNAVDFLGERKKIDVMKVDYSRENVASHLQVTFLIY

SEQ ID NO: 36 - protein, 50 aa, Sorghum bicolor
KKKIWWTNPLQDLFLQAIQHIGDKKKIAIMNVPYTRENVASHLQKYRLFV

SEQ ID NO: 37 - protein, 50 aa, Sorghum bicolor
KVKVDWTPELHRRFVQAVEQLGDKSRIEIMGTDCTRHNIASHLQKYRSHR

SEQ ID NO: 38 - protein, 50 aa, Sorghum bicolor
NPDLVWTNRLQLVFDDAVVRLGFSKAIELISEEGTGDQIRSHLQVLRDRQ

SEQ ID NO: 39 - protein, 50 aa, Sorghum bicolor
KKGVPWTEEEHRMFLLGLQKLGDWRGIRNYVISRTPTQVASHAQKYFIRQ

SEQ ID NO: 40 - protein, 50 aa, Sorghum bicolor
KQRERWTEDEHERFLEALRLYGAWQRIEEHIGTKTAVQIRSHAQKFFTKL

SEQ ID NO: 41 - protein, 50 aa, Sorghum bicolor
KSRESWTEGEHDKFLEALQLFDDWKKIEDFVGSKTVIQIRSHAQKYFLKV

SEQ ID NO: 42 - protein, 50 aa, Sorghum bicolor
KQRRCWSSQLHRRFLNALQHLGHVKQIEFMKVDGTNDEVKSHLQKYRLHT

SEQ ID NO: 43 - protein, 50 aa, Sorghum bicolor
KARLRWSSDLHDCFVNAVEKLGNKKSVEAMEVEGALHHVKSHLQKFRLGK

SEQ ID NO: 44 - protein, 409 aa, Sorghum bicolor
MNYNFSSNALDEEEVAGRGGEGGSCAAAPAWARPCDGCRAAPSVVYCHADAAYLCASCDRVHAANRVASHERVRVCEACERAPAVLACRADAAALCVVCDAQVHSANPLAGRHQRVPVLPLPVAAIPAASVLAEAAATAVAVGDKQEEEVDSWLLLTNTKDPVSDNNNCNCSSSSNNNISSSNTSTFYADVDEYFDLVGYNSYCDNHINSNPKQYGMQERQQQQQLLLQKEFGDKEGSEHVVPASQVAMANEQQQSGYGVIGVEQAASMTAAVSAYTDSITNSISFSSSMEVGIVPDNMATTTDMPNSGILLTPAEAISLFSSGSSLQMPLHLTSMDREARVLRYKEKKKSRKFAKTIRYATRKTYAEARPRIKGRFAKRSSDMEIEVDQMFSSAALSSDGSYGTVLWF

SEQ ID NO: 45 - protein, 429 aa, Sorghum bicolor
MTPSVTPWIPLAPTLLLVLCTGLAALWIHHCNFMDYNFDTSVLDEDVAGRGGREGSCPPAWARACDGCRAAPSVVYCHADTAYLCASCNSRVHAANRVASRHERVRVCEACECAPAVLACRADAAALCAACDAQVHSANPLAGRHQRVPVLPLPAAAVPAASVLAEASAATAAAVAGDKDEEVDSWLLLTKDPDDDDKNHNCSSNNNNISSNTSTFYADVDEYFDLVGYSSYCDNHINSNTKQYGMQEQQLLLHKEFGDKEGSEYVVPSQVGQQQSGYHRVIGTEQAASMTPGVSAYTDSISNSISFSSSMEVGIVPDNMATTDMPSSGILLTPAGAISLFSSGPPLQMPLHLASMDREARVLRYREKKKSRKFEKTIRYATRKTYAEARPRIKGRFAKRSSDMDVEVDQMFSAAALSSDGSYGTVPWF

SEQ ID NO: 46 - protein, 395 aa, Sorghum bicolor
MNYNFGGNVFDQEVGVGGEGGGGGEGSGCPWARPCDGCRAAPSVVYCRADAAYLCASCDARVHAANRVASRHERVRVCEACERAPAALACRADAAALCVACDVQVHSANPLPAITIPATSVLAEAVVATATVLGDKDEEVDSWLLLSKDSDNNNNNNNNNDNDNNDNNNSNSSNNGMYFGEVDEYFDLVGYNSYYDNRIENNQDRQYGMHEQQEQQQQQQEMQKEFAEKEGSECVVPSQITMLSEQQHSGYGVVGADQAASMTAGVSAYTDSISNSISFSSMEAGIVPDSTVIDMPNSRILTPAGAINLFSGPSLQMSLHFSSMDREARVLRYREKKKARKFEKTIRYETRKAYAEARPRIKGRFAKRSDVQIEVDQMFSTAALSDGSYGTVPWF

SEQ ID NO: 47 - protein, 383 aa, Sorghum bicolor
MNCVSNGTVYEEAVGREGRWARLCDGCCTVPSVVYCRADSAYLCASCDAQIHAANRVASRHERVLLSEAYKHAPVMLDCHADAAALCAAYEAQVHYANLLTVMHQRMPVVSHPAVAIPPVSLFAEAEATAPVLGRKEEDTSWLLLSKDSDNHNRSGNNSSTSSSSQYFGEVDQYFDLVGYNSYYDSHMSNQEQYVMQEQQHLQQMQKEYAEQQMQKEYVENEGSECIVPSQSTIVRRPHQSGYAPLVGAEQAASATAGASAYTDSVNNSISFSMEAGIVPDNTVQSSILRPAGAIGLFSSPSLQTPLHFSSKEREARVLRYKEKKKSRKFEKTTRYATRKAYAEARPRIKGRFAKRSDAEMEVDQTFSTAALSDSSYSTVPWF

SEQ ID NO: 48 - protein, 373 aa, Sorghum bicolor
MLKQESNDIGSGENNRARPCDTCRSNACTVYCHADSAYLCMSCDAQVHSANRVASRHKRVRVCESCERAPAAFLCEADDASLCTACDSEVHSANPLARRHQRVPILPISGNSFSSMTTTHHQSEKTMTDPEKRLVVDQEEGEEGDKDAKEVASWLFPNSDKNNNNQNNGLLFSDEYLNLVDYNSSMDYKFTGEYSQHQQNCSVPQTSYGGDRVVPLKLEESRGHQCHNQQNFQFNIKYGSSGTHYNDNGSINHNAYISSMETGVVPESTACVTTASHPRTPKGTVEQQPDPASQMITVTQLSPMDREARVLRYREKRKTRKFEKTIRYASRKAYAEIRPRVNGRFAKREIEAEEQGFNTMLMYNTGYGIVPSF

(19) United States
(12) Patent Application Publication
(10) Pub. No.: US 2016/0135415 A1
(43) Pub. Date: May 19, 2016

(54) SORGHUM HYBRIDS WITH DELAYED FLOWERING TIMES

(71) Applicant: The Texas A&M University System, College Station, TX (US)

(72) Inventors: John E. Mullet, College Station, TX (US); Daryl Morishige, College Station, TX (US); William L. Rooney, College Station, TX (US)

(21) Appl. No.: 14/944,053

(22) Filed: Nov. 17, 2015

Related U.S. Application Data
(60) Provisional application No. 62/081,507, filed on Nov. 18, 2014.

(51) Int. Cl. A01H 5/10; C10L 8/00; A01H 1/04; C12Q 1/68; A01H 1/02

(52) U.S. Cl. CPC A01H 5/10; C12Q 1/6895; A01H 1/02; A01H 1/04; C10L 8/00; C12Q 2600/13; C12Q 2600/156; C10L 2200/0469

(57) ABSTRACT

Methods and compositions for the production of sorghum hybrids with selected or delayed flowering times are provided. In accordance with the invention, a substantially continual and high-yield harvest of sorghum is provided. Improved methods of seed production are also provided.

### CROSS REFERENCE TO RELATED APPLICATIONS

This application claims priority to U.S. Provisional Application No. 62/081,507, filed Nov. 18, 2014, which is herein incorporated by reference in its entirety.

### FIELD OF THE INVENTION

The present invention relates to the field of agricultural biotechnology. More specifically, the invention relates to methods for producing sorghum plants with delayed or defined flowering times.

### INCORPORATION OF SEQUENCE LISTING

A sequence listing contained in the file named “TAMC032US_ST25.txt” which is 93,335 bytes (measured in MS-Windows®) and created on Nov. 17, 2015, comprises 8 nucleotide sequences, is filed electronically herewith and incorporated by reference in its entirety.

### BACKGROUND OF THE INVENTION

Optimal regulation of the timing of floral transition in sorghum crops is critically important for reproductive success and crop yield. While sorghum lines have been selected for use as sources of grain, sugar, forage, or biomass, there remains a need in the art for sorghum varieties with improved flowering time traits and methods for their production. Efforts to identify sorghum lines exhibiting desirable flowering time traits have been complicated by the many factors which contribute to flowering time, including the stage of plant development, signals from the photoperiod, temperature, and growing location. Moreover, there has previously been a lack of understanding of the genetic factors controlling flowering time, resulting in difficulties in identifying and using alleles conferring desirable flowering time traits. Without increased knowledge of the particular alleles involved in flowering time in sorghum and molecular markers for identifying and tracking these alleles during plant breeding, it may not be practical to attempt to produce certain new genotypes of crop plants due to such challenges.

### SUMMARY OF THE INVENTION

In one aspect, the invention provides methods of obtaining sorghum plants exhibiting delayed or early flowering time comprising: a) providing a population of sorghum plants; b) detecting in said population a plant comprising a delayed flowering time allele at a polymorphic locus in, or genetically linked to, a chromosomal segment between approximately 19.2 Mbp and 22.0 Mbp on chromosome 1; or at a polymorphic locus in, or genetically linked to, a chromosomal segment between approximately 6.2 Mbp and 8.2 Mbp on chromosome 1; or at a polymorphic locus in, or genetically linked to, a chromosomal segment between approximately 48.1 Mbp and 50.3 Mbp on chromosome 8; or at a polymorphic locus in, or genetically linked to, a chromosomal segment between approximately 10.1 Mbp and 13.7 Mbp on chromosome 10; and c) selecting said plant from said population based on the presence of said allele; wherein said plant exhibits delayed or early flowering compared to a control plant lacking said delayed flowering time allele. In some embodiments, said polymorphic locus is in or genetically linked to SbEHD1 or SbCO. In certain embodiments, said SbEHD1 gene encodes an Sbehd1 protein comprising a mutation at a position homologous to amino acid 189, 201, 202, or 269 of SEQ ID NO: 4 relative to SEQ ID NO: 4. In further embodiments, said SbCO gene encodes an SbCO protein comprising a mutation at a position homologous to amino acid 106 of SEQ ID NO: 8 relative to SEQ ID NO: 8. In some embodiments, step (a) of providing comprises crossing a first sorghum plant comprising a delayed flowering time allele with a second sorghum plant to produce a population of sorghum plants. In further embodiments, said population of sorghum plants comprises selfing or backcrossing. In yet further embodiments, step (b) of detecting comprises the use of an oligonucleotide probe.

In another aspect, the invention provides methods of producing sorghum plants exhibiting delayed or early flowering time comprising: a) crossing a first sorghum plant comprising a delayed flowering time allele with a second sorghum plant of a different genotype to produce one or more progeny plants; and b) selecting a progeny plant based on the presence of said allele at a polymorphic locus in, or genetically linked to, a chromosomal segment between approximately 19.2 Mbp and 22.0 Mbp on chromosome 1; or at a polymorphic locus in, or genetically linked to, a chromosomal segment between approximately 6.2 Mbp and 8.2 Mbp on chromosome 1; or at a polymorphic locus in, or genetically linked to, a chromosomal segment between approximately 48.1 Mbp and 50.3 Mbp on chromosome 8; or at a polymorphic locus in, or genetically linked to, a chromosomal segment between approximately 10.1 Mbp and 13.7 Mbp on chromosome 10; wherein said allele confers delayed or early flowering time compared to a plant lacking said allele. In some embodiments, said polymorphic locus is in or genetically linked to SbEHD1 or SbCO. In further embodiments, said SbEHD1 gene encodes an Sbehd1 protein comprising a mutation at a position homologous to amino acid 189, 201, 202, or 269 of SEQ ID NO: 4 relative to SEQ ID NO: 4. In yet further embodiments, said SbCO gene encodes an SbCO protein comprising a mutation at a position homologous to amino acid 106 of SEQ ID NO: 8 relative to SEQ ID NO: 8. In certain embodiments, step b) of selecting further comprises selecting a progeny plant which is homozygous for said allele. In some embodiments, the methods of the invention further comprise: c) crossing said progeny plant with itself or a second plant to produce one or more further progeny plants; and d) selecting a further progeny plant comprising said allele. In certain embodiments, step (d) of selecting comprises marker-assisted selection. In further embodiments, said further progeny plant is an F2-F7 progeny plant. In yet further embodiments, producing the progeny plant comprises selfing or backcrossing. In certain embodiments, backcrossing comprises from 2-7 generations of selfing or backcrossing. In further embodiments, selfing or backcrossing comprises marker-assisted selection. In yet further embodiments, selfing or backcrossing comprises marker-assisted selection in at least two generations. In some embodiments, selfing or backcrossing comprises marker-assisted selection in all generations. In certain embodiments, said first sorghum plant is an inbred or a hybrid. In further embodiments, said second sorghum plant is an agronomically elite sorghum plant, for example BTx642. In yet further embodiments, said agronomically elite sorghum plant is an inbred or a hybrid.

In further aspects, the invention provides plants, plant parts, and seeds produced by the methods provided herein.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a sorghum flowering time pathway according to the present invention.

FIG. 2 shows flowering time regulation in sorghum in a short day environment according to the present invention.

FIG. 3 shows quantitative trait loci (QTL) associated with flowering time in a BTx642/Tx7000 recombinant inbred line (RIL) population. Flowering time QTL are shown for RIL populations were grown under long day (LD) greenhouse conditions (top panel), field conditions (middle panel), and short day (SD) greenhouse conditions (bottom panel). Permutation tests were carried out to identify 95% confidence thresholds and significant threshold of LOD score is presented as a horizontal line. Candidate genes within the identified QTL regions are noted above several peaks.

FIG. 4 shows an alignment of EHD1 (Sb01g019980) mRNA sequences. Silent mutations and missense mutations are shown in gray.

FIG. 5 shows an alignment of EHD1 (Sb01g019980) protein sequences. A conserved signal receiver domain and a conserved Myb-like DNA binding domain are shown in dark gray. Amino acid changes are shown in light gray.

FIG. 6 shows an alignment of a conserved signal receiver domain found within the Ehd1 protein sequence identified by the present invention. The domain was originally thought to be unique to bacteria, and has recently been identified in eukaryotes. This domain receives a signal from the sensor partner in a two-component system, and contains a phosphoacceptor site that is phosphorylated by histidine kinase homologs, usually found N-terminal to a DNA binding effector domain. The domain forms homodimers. SEQ ID NOs are shown in parentheses.

FIG. 7 shows an alignment of a conserved myb-like DNA-binding domain within the Ehd1 protein sequence identified by the present invention. The domain is a DNA-binding domain restricted to (but common in) plant proteins, many of which also contain a response regulator domain. The domain appears related to the Myb-like DNA-binding domain described by pfam00249. It is distinguished in part by a well-conserved motif SH[AL]QKY[RF] at the C-terminal end of the motif. SEQ ID NOs are shown in parentheses.

FIGS. 8A and 8B shows an alignment of CONSTANS homologs, including the sorghum CONSTANS homolog (SbCO) identified by the present invention. (FIG. 8A) shows the protein structure of SbCO with the domains characteristic of CONSTANS-like gene families: B-box1, B-box2, and CCT domain are boxed. Asterisks above the His106Tyr mutation identified by the present invention indicate that this functional mutation was also identified in rice and Arabidopsis. (FIG. 8B) shows multiple sequence alignments of CO homologs from sorghum (Sb10g010050, SbCO), maize (GRMZM2G405368_T01, conz1), rice (Os06g16370, OsHd1), barley (AF490468, HvCO1) and Arabidopsis (AT5G15850, AtCO). The sorghum sequence used for alignment was derived from BTx623 (SbCO-1). Protein residues conserved among all 5 species are underscored by asterisks. Amino acid residues underscored by a colon indicate residues of strong conserved properties, while residues underscored by a period indicate residues with more weakly similar properties. One amino acid substitution distinguishes BTx623 (SbCO-1) and Tx7000 (SbCO-2) (marked with a light gray arrow). Unique amino acid substitutions that distinguish BTx623 and BTx642 (Sbco-3) are marked with black arrows (tolerant) and a light gray arrow (intolerant).

### DETAILED DESCRIPTION

Regulation of flowering time in sorghum is essential for achieving optimal crop yield and reproductive success. Growth duration is a determinant of biomass yield, and therefore non-flowering plants or plants that flower late in a growing season are desirable for accumulating biomass before vegetative growth ceases at flowering. It is estimated that late or non-flowering sorghum is capable of generating more than two times the biomass accumulated by photoperiod insensitive early flowering sorghum throughout the growing season under good growth conditions.

Efforts to identify or produce sorghum lines with delayed or early flowering have previously been hindered by a limited understanding of the genetic loci controlling flowering time and a lack of available markers for detecting and tracking flowering time alleles in plants. This lack of understanding has been further complicated by polygenic inheritance of flowering time traits. Therefore, a need for sorghum plants exhibiting desirable flowering time traits remains.

Despite the many obstacles to identifying the genetic loci regulating flowering time in sorghum, Applicants were able to identify quantitative trait loci (QTL), candidate genes, and genetic markers associated with favorable flowering time alleles in sorghum plants. The inventors were further able to develop improved breeding methods using the genetic markers of the present invention for producing sorghum plants with desirable flowering time characteristics. The invention therefore represents a significant advance in the art.

In some embodiments, the invention provides QTL associated with favorable flowering time traits on chromosomes 1, 6, 8, and 10 of the sorghum genome. The invention further provides chromosomal segments between approximately 19.2 Mbp and 22.0 Mbp on chromosome 1 or between approximately 10.1 Mbp and 13.7 Mbp on chromosome 10 that are associated with the regulation of flowering time, and genetic markers within or genetically linked to these segments. The invention also provides coding sequences SbEHD1 and SbCO within the sorghum genome associated with the regulation of flowering time. In further embodiments, the invention provides and identifies novel single polynucleotide polymorphisms (SNPs) that allow for the identification and tracking of flowering time alleles in plants. In certain embodiments, polymorphisms provided by the invention are located at a position corresponding to position 2573, 5636, 5672, 5676, or 6391 of SEQ ID NO: 1; a position corresponding to position 375, 778, 814, 818, or 1020 of SEQ ID NO: 2, or at a position corresponding to position 162, 565, 601, 605, or 807 of SEQ ID NO: 3. In other embodiments, polymorphisms provided by the invention are located at a position corresponding to position 605 of SEQ ID NO: 5; a position corresponding to position 605 of SEQ ID NO: 6; or at a position corresponding to position 316 of SEQ ID NO: 7. In some embodiments, polymorphisms identified by the invention include polymorphisms resulting in a mutation at a position homologous to amino acid 189, 201, 202, or 269 of SEQ ID NO: 4 relative to SEQ ID NO: 4. In other embodiments, polymorphisms identified by the invention include polymorphisms resulting in mutation at a position homologous to amino acid 106 of SEQ ID NO: 8 relative to SEQ ID NO: 8.

In further embodiments, the invention provides improved breeding methods utilizing the novel QTL and markers disclosed herein for the production of sorghum plants with favorable flowering time traits. Without the knowledge of the QTL or specific polymorphic loci associated with flowering time traits provided by the present invention, conventional breeding methods would require prohibitively large segregating populations for progeny screens, and phenotyping in environments with short day lengths. Marker-assisted selection (MAS) is therefore essential for the effective production of plant lines with optimal flowering time traits. The present invention enables MAS by providing improved and validated markers for detecting genotypes associated with desirable flowering time characteristics and eliminates the need to grow large populations of plants to anthesis in short day environments in order to observe the phenotype.

### I. Flowering Time in Sorghum Crops

Biomass yield is one of the most important attributes of a biomass or bioenergy crop designed to accumulate ligno-cellulose and fermentable sugars for conversion to biofuels or bioenergy. Growth duration is a determinant of biomass yield, therefore non-flowering plants or plants that flower late in a growing season accumulate the most biomass assuming environmental conditions allow yield potential to be expressed. Use of non-flowering or delayed flowering plants also prevents propagation of seed from elite hybrids (genotype protection) and blocks transgene flow in cases where transgenic plants are used commercially. Further, the production of sorghum hybrids that flower and accumulate elevated amounts of sugar at different times in the growing season may find use in industry since these hybrids allow staggered harvest times during the season. This maximizes yield across the growing season, allows for improved planning of harvest time and extends the duration of biorefinery operation. In particular embodiments, R-line (males) and A/B-lines (females) are provided that, when crossed, will produce hybrids that flower at different times or not at all during a growing season.

Most sorghum hybrids will flower in 60-90 days when grown in short day (SD) environments, depending on temperature. The present invention provides novel QTL and genetic markers that enable production of sorghum hybrids that flower in 90 to 120 days or later when grown in short day environments. The delayed flowering of hybrids will be observed when plants are grown near the equator and in the spring or fall when plants are grown at higher latitudes. The delayed or early flowering associated with the QTL of the invention can lead directly to increased yield in sorghum crops.

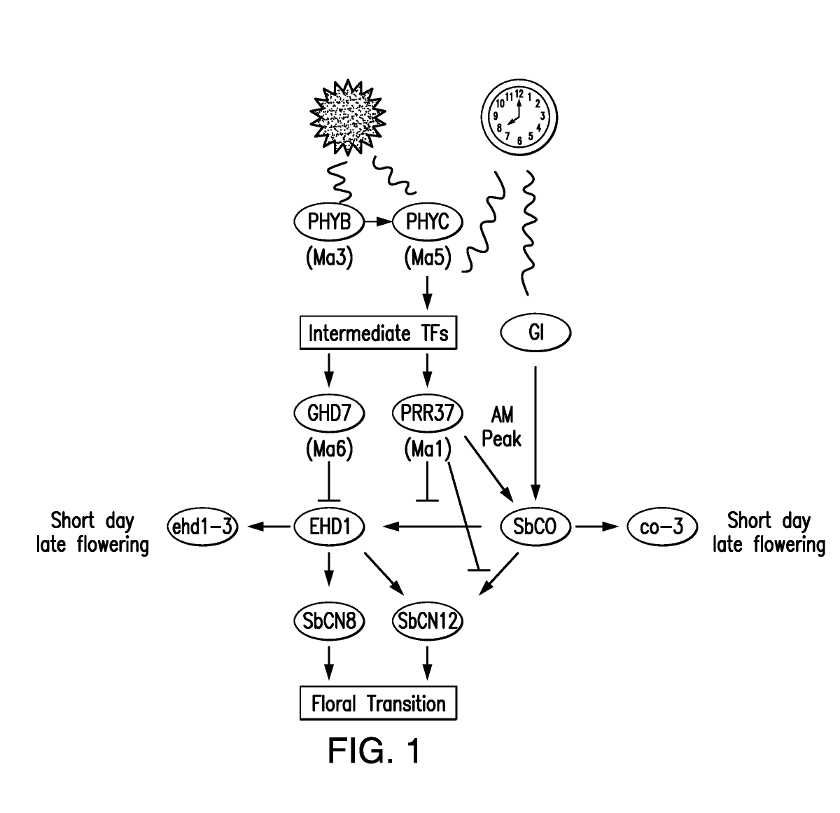

The novel QTL of the invention can be combined with additional flowering time loci such as CN8, CN12, SbCDF1, EHD3, and ELF3, PRR37, GHD7 and PHYC, that mediate photoperiod sensitive or insensitive flowering in short or long days to provide plants exhibiting optimal flowering time for the desired use and growing region. Ma1, Ma5, and Ma6 involved in delayed flowering time in long days and the EHD1 and CO genes provided by the present invention involved in delayed flowering in short days, are shown in the flowering time regulatory pathway shown in FIG. 1. This figure indicates that EHD1 and CO are activators of CN8 and CN12, genes that encode FT-like ‘florigens’ that induce formation of flowers. Sorghum and other grasses modify flowering time by regulating the expression of specific genes in the PEBP family (i.e., SbCN8, SbCN12) that encode florigens that move from leaves to the shoot apical meristem where they induce formation of flowers. In maize, activation of ZCN8/12, and in sorghum, activation of the orthologs SbCN8/12 in leaves sends a signal (florigen, a protein) to the shoot apical meristem that induces transition from vegetative growth to the flowering program.

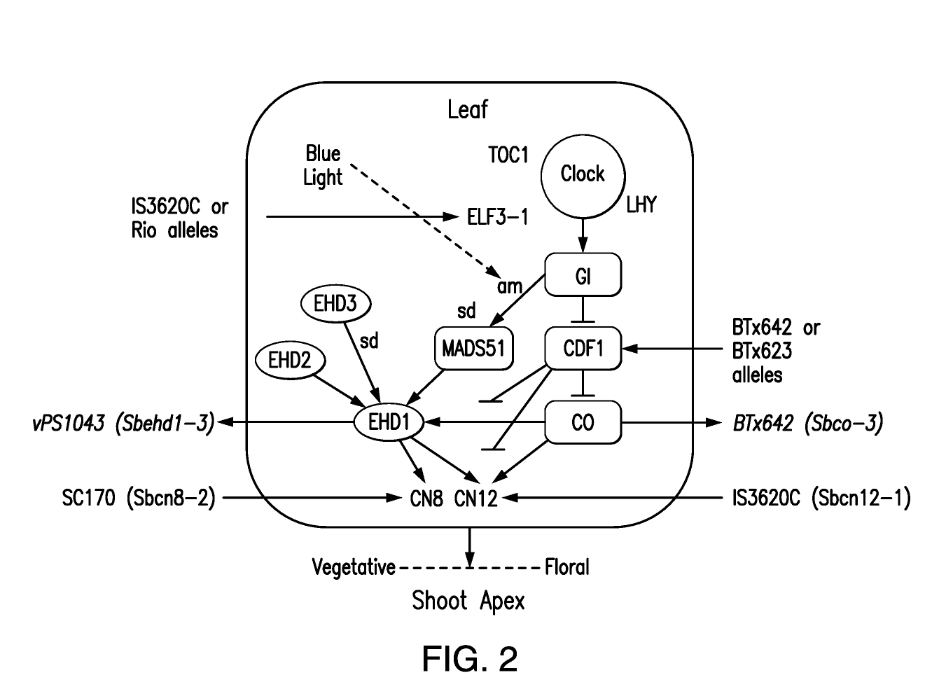

FIG. 2 shows the main regulators of flowering in short days as provided in the present invention. In short days, expression of the repressors Ma1 (PRR37) and Ma6 (GHD7) is reduced significantly and as a consequence these genes have minimal influence on flowering time. Instead, the newly identified genes provided by the invention, including EHD1 and CO, are important for the regulation of flowering in short days. As provided by the invention, EHD1 strongly activates CN8 and also increases expression of CN12. CO activates EHD1 and also activates CN12. Expression of SbCN8 and SbCN12 in leaves is activated by SbCO (CONSTANS) and SbEhd1 (Early Heading Date 1) discovered by Applicants. SbCO and SbEhd1 expression is regulated by day length, development, and other factors so that the timing of floral induction for a given genotype is optimized for plant reproduction. CO expression is regulated by the Clock and genes that mediate Clock regulation of CO (G1, CDF1, etc.). The activity of genes such as EHD2, EHD3, MADS50/51, ELF3, CDF1, and GI also regulate EHD1 thereby altering flowering time in short days. In short days, EHD1 is activated by EHD2, EHD3, and MADS51. MADS51 is regulated by the blue light signaling pathway involving several factors (CRY, ELF3) and the Clock.

The present invention overcomes problems with current sorghum production technologies in providing inbred varieties that flower at desired maturation times. By manipulation of maturation times in accordance with the invention, hybrids providing a substantially high-yield harvest can be designed for harvest throughout a growing season. In one embodiment of the invention, such methods permit the efficient delivery of biofuel sorghum to a biofuel biorefinery without substantial interruption of availability of feedstock for biofuel production between harvests. By providing multiple inbreds having selected genetic contributions for maturity, the seed of such hybrids can be produced, and numerous different desired maturation times may be incorporated into selected hybrid germplasm.

In another aspect, a system is provided for the production of biofuel comprising harvesting biomass from a plurality of sorghum hybrids produced according to a method of the invention and producing biofuel from the biomass, comprised of lignocellulose and fermentable sugars, wherein harvesting is staggered to provide a substantially continuous supply of the biomass. In the system, the plurality of sorghum hybrids may be planted substantially simultaneously with one another. In one embodiment, the plurality of sorghum hybrids comprises hybrids with at least 3, 4, or 5 different dates of maturity.

### II. Quantitative Trait Loci

A quantitative trait locus (QTL) is a chromosome interval which may comprise a single gene or multiple genes associated with a genetic trait, such as flowering time. Each interval comprising a QTL comprises at least one gene conferring a given trait, however knowledge of how many genes are in a particular interval is not necessary to make or practice the invention, as such an interval will segregate at meiosis as a linkage block unless recombination occurs within the block. In accordance with the invention, a chromosomal interval comprising a QTL may therefore be readily introgressed and tracked in a given genetic background using the methods and compositions provided herein.

Identification of chromosomal intervals and QTL is therefore beneficial for detecting and tracking genetic traits, such as flowering time traits, in plant populations. In some embodiments, this is accomplished by identification of markers linked to a particular QTL. The principles of QTL analysis and statistical methods for calculating linkage between markers and useful QTL include penalized regression analysis, ridge regression, single point marker analysis, complex pedigree analysis, Bayesian MCMC, identity-by-descent analysis, interval mapping, composite interval mapping (CIM), and Haseman-Elston regression. QTL analyses may be performed with the help of a computer and specialized software available from a variety of public and commercial sources known to those of skill in the art.

In some embodiments, the invention provides a chromosomal interval comprising a QTL associated with flowering time in plants. The invention also provides multiple markers associated with the QTL provided herein. The present invention further provides a plant comprising alleles of the chromosome intervals linked to flowering time described herein, or fragments and complements thereof. Plants provided by the invention may be homozygous or heterozygous for such alleles.

Accordingly, the compositions and methods of the present invention can be utilized to guide MAS or breeding sorghum varieties or hybrids with a desired complement (set) of allelic forms of genes that regulate flowering time present in chromosome intervals associated with desirable flowering time traits. Any of the disclosed marker alleles can be introduced into a sorghum line via introgression, by traditional breeding (or introduced via transformation, or both) to yield sorghum plants with desired flowering time traits.

Thus, the invention permits one skilled in the art to detect the presence or absence of flowering time genotypes in the genomes of sorghum plants as part of a MAS program. In one embodiment, a breeder ascertains the genotype at one or more markers for a parent with favorable flowering time traits and for a parent lacking the favorable trait. A breeder can then reliably track the inheritance of the flowering time alleles through subsequent populations derived from crosses between the two parents by genotyping offspring with the markers used on the parents and comparing the genotypes at those markers with those of the parents. Progeny that share genotypes with a parent can be reliably predicted to express the parent phenotype. Thus, the laborious, inefficient, and potentially inaccurate process of manually phenotyping the progeny is avoided.

By providing the positions in the sorghum genome of the flowering time genes located within specified genomic intervals and associated markers within those intervals, the invention also allows one skilled in the art to identify and use other markers within the intervals disclosed herein or linked to the intervals disclosed herein. Having identified such regions, these markers can be readily identified from public linkage maps.

The choice of markers actually used to practice the invention is not limited and can be any marker that is genetically linked to the intervals containing specified flowering time gene alleles as described herein, which includes markers mapping within the intervals. In certain embodiments, the invention further provides markers closely genetically linked to, or within approximately 0.5 cM of, the markers provided herein and chromosome intervals whose borders fall between or include such markers, and including markers within approximately 0.4 cM, 0.3 cM, 0.2 cM, and about 0.1 cM of the markers provided herein. Furthermore, since there are many different types of marker detection assays known in the art, it is not intended that the type of marker detection assay used to practice this invention be limited in any way.

### III. Molecular Markers

A “marker,” “genetic marker,” “molecular marker,” or “marker locus” refers to a nucleotide sequence or encoded product thereof (e.g., a protein) used as a point of reference when identifying a genetic locus linked to a trait. A marker can be derived from genomic nucleotide sequences or from expressed nucleotide sequences (e.g., from a spliced RNA, a cDNA, etc.), or from an encoded polypeptide, and can be represented by one or more particular variant sequences, or by a consensus sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence.

“Marker” also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also know for the detection of expressed sequence tags (ESTs) and SSR markers derived from EST sequences and randomly amplified polymorphic DNA (RAPD).

PCR detection and quantification using dual-labeled fluorogenic oligonucleotide probes, commonly referred to as “TaqMan™” probes, can also be performed according to the present invention. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quenching dye by FRET. During the extension phase of PCR, the probe is cleaved by 5′ nuclease activity of the polymerase used in the reaction, thereby releasing the reporter from the oligonucleotide-quencher and producing an increase in reporter dye florescence emission intensity. TaqMan™ probes are oligonucleotides that have a label and a quencher, where the label is released during amplification by the exonuclease action of the polymerase used in amplification, providing a real time measure of amplification during synthesis. Therefore, selective hybridization and extension of TaqMan probes designed to detect different allelic sequences of a target gene or marker is detected by increased fluorescence emission of dye's released during PCR amplification. A variety of TaqMan™ reagents are commercially available, e.g., from Applied Biosystems as well as from a variety of specialty vendors such as Biosearch Technologies.

In one embodiment, the presence or absence of a molecular marker is determined simply through nucleotide sequencing of the polymorphic marker region. This method is readily adapted to high throughput analysis as are the other methods noted above, e.g., using available high throughput sequencing methods.

In alternative embodiments, the sequence of a nucleic acid comprising the marker locus of interest can be stored in a computer. The desired marker locus sequence or its homolog can be identified using an appropriate nucleic acid search algorithm as provided by, for example, in such readily available programs as BLAST, or even simple word processors.

“Linkage”, or “genetic linkage,” is used to describe the degree with which one marker locus is associated with another marker locus or some other locus (for example, a flowering time locus). A marker locus may be located within a locus to which it is genetically linked. As used herein, linkage can be between two markers, or alternatively between a marker and a mutation that causes a phenotype. A marker locus may be genetically linked to a trait, and in some cases a marker locus genetically linked to a trait is located within the allele conferring the trait. A marker may also be causative for a trait or phenotype, for example a causative polymorphism. The degree of linkage of a molecular marker to a phenotypic trait can be measured, e.g., as a statistical probability of co-segregation of that molecular marker with the phenotype.

As used herein, “closely linked” means that the marker or locus is within about 10 cM, for instance within about 5 cM, about 1 cM, about 0.5 cM, or less than 0.5 cM of the identified locus associated with a trait.

Linkage analysis is used to determine which polymorphic marker allele demonstrates a statistical likelihood of co-segregation with a given phenotype. Following identification of a marker allele that co-segregates with causative sequence variants that affect the phenotype, it is possible to use this marker for rapid, accurate screening of plants for the allele without the need to grow the plants through their life cycle and await phenotypic evaluations, and furthermore, permits genetic selection for the particular allele even when the molecular identity of the causative sequence variant underlying a QTL is unknown. Tissue samples can be taken, for example, from the endosperm, embryo, or mature/developing plant and screened with the appropriate molecular marker to rapidly determine determined which progeny contain the desired genetics. Linked markers also remove the impact of environmental factors and epistatic interactions that can often influence phenotypic expression.

### IV. Marker Assisted Selection

“Introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background.

A primary motivation for development of molecular markers in crop species is the potential for increased efficiency in plant breeding through MAS. Genetic markers are used to identify plants that contain a desired genotype at one or more loci, and that are expected to transfer the desired genotype, along with a desired phenotype to their progeny. Genetic markers can be used to identify plants containing a desired genotype at one locus, or at several unlinked or linked loci (e.g., a haplotype), and that would be expected to transfer the desired genotype, along with a desired phenotype to their progeny, in some instances inbreds or hybrids. The present invention provides the means to identify plants that carry various flowering time traits.

Identification of plants or germplasm that include a marker locus or marker loci linked to a trait or traits provides a basis for performing MAS. Plants that comprise favorable markers or favorable alleles are selected for, while plants that comprise markers or alleles that are unfavorable can be selected against. Desired markers and/or alleles can be introgressed into plants having a desired (e.g., elite or exotic) genetic background to produce an introgressed plant or germplasm. In some aspects, it is contemplated that a plurality of markers are sequentially or simultaneous selected and/or introgressed. The combinations of markers that are selected for in a single plant is not limited, and can include any combination of markers disclosed herein or any marker linked to the markers disclosed herein, or any markers located within the QTL intervals defined herein.

### V. Introgression of Flowering Time Alleles Using MAS

In some embodiments, a first sorghum plant or germplasm (the donor) can be crossed with a second sorghum plant or germplasm (the recipient) to create an introgressed sorghum plant or germplasm as part of a breeding program designed to confer desired flowering time traits to the recipient sorghum plant or germplasm. In some aspects, one or more flowering time loci can be conferred to the recipient, which can be qualitative or quantitative trait loci. In another aspect, a transgene can be conferred to the recipient.

The introgression of one or more desired loci from a donor line into another is achieved via a cross followed by selfing or one or more backcrosses to a recurrent parent accompanied by selection to retain one or more flowering time loci from the donor parent. Markers associated with flowering time are assayed in progeny and those progeny with one or more favorable flowering time markers are selected for advancement. In another aspect, one or more markers can be assayed in the progeny to select for plants with the genotype of the agronomically elite parent. This invention anticipates that trait introgression activities will require more than one generation, wherein progeny are crossed to the recurrent (agronomically elite) parent or selfed. Selections are made based on the presence of one or more flowering time markers and can also be made based on the recurrent parent genotype, wherein screening is performed on a genetic marker and/or phenotype basis. In another embodiment, markers of this invention can be used in conjunction with other markers, ideally at least one on each chromosome of the sorghum genome, to track the introgression of flowering time loci into a recipient germplasm.

In some embodiments of the invention, the SbCO or SbEhd1 alleles having reduced or absent activity provided by the invention can be used to construct R-lines (pollinators) and A/B-lines (seed parents) useful for the production of hybrid seed and hybrid plants that flower later in short days. In other embodiments of the invention, R-lines and A/B-lines comprising the SbCO or SbEhd1 alleles further comprise other regulators of CO and Ehd1 in order to construct hybrids that flower even later and at intermediate times when grown in short day photoperiods. In further embodiments, R-lines and A/B-lines comprising the SbCO or SbEhd1 alleles further comprise alleles of SbPRR37, SbPHYC and SbGHD7 to create hybrids that have delayed or flowering in short days and delayed flowering in long days due to photoperiod sensitivity.

The invention therefore further provides methods of construction of sorghum R-line (pollinators) and A/B-line (seed parents) inbreds that enable production of sorghum hybrids with defined and delayed or early flowering times in short or long day environments. Hybrids with optimized flowering times in short day environments will enable improved commercial production of sweet sorghum and high biomass sorghum. The allele combinations can also be used to modify flowering in long day environments and can be deployed in conjunction with alleles that confer photoperiod sensitivity. It is further contemplated that the methods provided herein would be useful in producing other C4 grasses exhibiting delayed or early flowering in short and long days.

VI. Methods for Producing Plants with Favorable Flowering Time Traits

Certain embodiments of the present invention provide sorghum genotypes that contain flowering time alleles that in combination delay flowering in part by modifying photoperiod sensitivity. In one embodiment of the invention, complementary dominant/recessive alleles of genes that control photoperiod sensitivity are present in R-lines (male) and A/B-lines (female). In this way parental R- and A/B lines may be bred to produce plants that flower within a desired timeframe to enable hybrid seed production. Such parental lines can be crossed to produce hybrids and progeny may be propagated easily, including for production of hybrid seed.

The invention further provides methods for the constructions of R-lines and A/B-lines comprising the SbCO or SbEhd1 alleles of the present invention, and further comprising other alleles associated with regulation of flowering time. In some embodiments, the other alleles comprise one or more of SbCN8, SbCN12, SbCDF1, SbEHD3, and SbELF3, SbPRR37, SbGHD7 and SbPHYC. The invention therefore provides inbred lines and hybrid lines produced therefrom which exhibit an array of desired flowering time traits.

### VII. Definitions

The definitions and methods provided define the present invention and guide those of ordinary skill in the art in the practice of the present invention. Unless otherwise noted, terms are to be understood according to conventional usage by those of ordinary skill in the relevant art. Examples of resources describing many of the terms related to molecular biology used herein can be found in in Alberts et al., Molecular Biology of The Cell, 5^{th} Edition, Garland Science Publishing, Inc.: New York, 2007; Rieger et al., Glossary of Genetics: Classical and Molecular, 5th edition, Springer-Verlag: New York, 1991; King et al, A Dictionary of Genetics, 6th ed., Oxford University Press: New York, 2002; and Lewin, Genes Icorn, Oxford University Press: New York, 2007. The nomenclature for DNA bases as set forth at 37 CFR §1.822 is used.

“Adjacent”, when used to describe a nucleic acid molecule that hybridizes to DNA containing a polymorphism, refers to a nucleic acid that hybridizes to DNA sequences that directly abut the polymorphic nucleotide base position. For example, a nucleic acid molecule that can be used in a single base extension assay is “adjacent” to the polymorphism.

“Allele” refers to an alternative nucleic acid sequence at a particular locus; the length of an allele can be as small as 1 nucleotide base, but is typically larger. For example, a first allele can occur on one chromosome, while a second allele occurs on a second homologous chromosome, e.g., as occurs for different chromosomes of a heterozygous individual, or between different homozygous or heterozygous individuals in a population. A favorable allele is the allele at a particular locus that confers, or contributes to, an agronomically desirable phenotype, or alternatively, is an allele that allows the identification of susceptible plants that can be removed from a breeding program or planting. A favorable allele of a marker is a marker allele that segregates with the favorable phenotype, or alternatively, segregates with susceptible plant phenotype, therefore providing the benefit of identifying phenotypes in plants. A favorable allelic form of a chromosome interval is a chromosome interval that includes a nucleotide sequence that contributes to superior agronomic performance at one or more genetic loci physically located on the chromosome interval. “Allele frequency” refers to the frequency (proportion or percentage) at which an allele is present at a locus within an individual, within a line, or within a population of lines. For example, for an allele “A,” diploid individuals of genotype “AA,” “Aa,” or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele. An allele positively correlates with a trait when it is linked to it and when presence of the allele is an indictor that the desired trait or trait form will occur in a plant comprising the allele. An allele negatively correlates with a trait when it is linked to it and when presence of the allele is an indicator that a desired trait or trait form will not occur in a plant comprising the allele.

“Crossed” or “cross” means to produce progeny via fertilization (e.g. cells, seeds or plants) and includes crosses between plants (sexual) and self fertilization (selfing).

“Elite line” means any line that has resulted from breeding and selection for superior agronomic performance. Numerous elite lines are available and known to those of skill in the art of plant breeding. An “elite population” is an assortment of elite individuals or lines that can be used to represent the state of the art in terms of agronomically superior genotypes of a given crop species. Similarly, an “elite germplasm” or elite strain of germplasm is an agronomically superior germplasm.

“Exogenous nucleic acid” is a nucleic acid that is not native to a specified system (e.g., a germplasm, plant, variety, etc.), with respect to sequence, genomic position, or both. As used herein, the terms “exogenous” or “heterologous” as applied to polynucleotides or polypeptides typically refers to molecules that have been artificially supplied to a biological system (e.g., a plant cell, a plant gene, a particular plant species or variety or a plant chromosome under study) and are not native to that particular biological system. The terms can indicate that the relevant material originated from a source other than a naturally occurring source, or can refer to molecules having a non-natural configuration, genetic location or arrangement of parts. In contrast, for example, a “native” or “endogenous” gene is a gene that does not contain nucleic acid elements encoded by sources other than the chromosome or other genetic element on which it is normally found in nature. An endogenous gene, transcript or polypeptide is encoded by its natural chromosomal locus, and not artificially supplied to the cell.

“Genetic element” or “gene” refers to a heritable sequence of DNA, i.e., a genomic sequence, with functional significance. The term “gene” can also be used to refer to, e.g., a cDNA and/or an mRNA encoded by a genomic sequence, as well as to that genomic sequence.

“Genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome. A “haplotype” is the genotype of an individual at a plurality of genetic loci. Typically, the genetic loci described by a haplotype are physically and genetically linked, i.e., on the same chromosome interval. The terms “phenotype,” or “phenotypic trait” or “trait” refers to one or more trait of an organism. The phenotype can be observable to the naked eye, or by any other means of evaluation known in the art, e.g., microscopy, biochemical analysis, genomic analysis, an assay, etc. In some cases, a phenotype is directly controlled by a single gene or genetic locus, i.e., a “single gene trait.” In other cases, a phenotype is the result of several genes.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells that can be cultured into a whole plant.

“Linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Linked loci co-segregate more than 50% of the time, e.g., from about 51% to about 100% of the time. The term “physically linked” is sometimes used to indicate that two loci, e.g., two marker loci, are physically present on the same chromosome. Advantageously, the two linked loci are located in close proximity such that recombination between homologous chromosome pairs does not occur between the two loci during meiosis with high frequency, e.g., such that linked loci cosegregate at least about 90% of the time, e.g., 91%, 92%, 93%, 94%, 95%, 96%, 97%, 98%, 99%, 99.5%, 99.75%, or more of the time.

“Locus” a chromosome region where a polymorphic nucleic acid, trait determinant, gene or marker is located. The loci of this invention comprise one or more polymorphisms in a population; i.e., alternative alleles are present in some individuals. A “gene locus” is a specific chromosome location in the genome of a species where a specific gene can be found.

“Marker Assay” means a method for detecting a polymorphism at a particular locus using a particular method, e.g. measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), microarray-based technologies, and nucleic acid sequencing technologies, etc. “Marker Assisted Selection” (MAS) is a process by which phenotypes are selected based on marker genotypes.

“Molecular phenotype” is a phenotype detectable at the level of a population of one or more molecules. Such molecules can be nucleic acids, proteins, or metabolites. A molecular phenotype could be an expression profile for one or more gene products, e.g., at a specific stage of plant development, in response to an environmental condition or stress, etc.

“Phenotype” means the detectable characteristics of a cell or organism which can be influenced by genotype.

“Plant” refers to a whole plant any part thereof, or a cell or tissue culture derived from a plant, comprising any of: whole plants, plant components or organs (e.g., leaves, stems, roots, etc.), plant tissues, seeds, plant cells, and/or progeny of the same. A plant cell is a biological cell of a plant, taken from a plant or derived through culture from a cell taken from a plant.

“Polymorphism” means the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and a tag SNP. A genetic marker, a gene, a DNA-derived sequence, a RNA-derived sequence, a promoter, a 5′ untranslated region of a gene, a 3′ untranslated region of a gene, microRNA, siRNA, a resistance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms.

A “population of plants” or “plant population” means a set comprising any number, including one, of individuals, objects, or data from which samples are taken for evaluation, e.g. estimating QTL effects. Most commonly, the terms relate to a breeding population of plants from which members are selected and crossed to produce progeny in a breeding program. A population of plants can include the progeny of a single breeding cross or a plurality of breeding crosses, and can be either actual plants or plant derived material, or in silico representations of the plants. The population members need not be identical to the population members selected for use in subsequent cycles of analyses or those ultimately selected to obtain final progeny plants. Often, a plant population is derived from a single biparental cross, but may also derive from two or more crosses between the same or different parents. Although a population of plants may comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population.

“Recombinant” in reference to a nucleic acid or polypeptide indicates that the material (e.g., a recombinant nucleic acid, gene, polynucleotide, polypeptide, etc.) has been altered by human intervention. The term recombinant can also refer to an organism that harbors recombinant material, e.g., a plant that comprises a recombinant nucleic acid is considered a recombinant plant.

“Transgenic plant” refers to a plant that comprises within its cells a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been altered by the presence of heterologous nucleic acid including those transgenic organisms or cells initially so altered, as well as those created by crosses or asexual propagation from the initial transgenic organism or cell. The term “transgenic” as used herein does not encompass the alteration of the genome (chromosomal or extrachromosomal) by conventional plant breeding methods (e.g., crosses) or by naturally occurring events such as random cross-fertilization, non-recombinant viral infection, non-recombinant bacterial transformation, non-recombinant transposition, or spontaneous mutation.

“Yield” is the culmination of all agronomic traits as determined by the productivity per unit area of a particular plant product of commercial value. “Agronomic traits,” include the underlying genetic elements of a given plant variety that contribute to yield over the course of growing season.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification of Flowering Time QTL

Flowering time QTL were mapped in a recombinant inbred line (RIL) population derived from a cross of BTx642 and Tx7000 genotypes (Yang, et al., 2014, the entirety of which is incorporated herein by reference). The genomes of BTx642 and Tx7000 were sequenced, and digital genotyping was used to create a high-resolution genetic map aligned to the genome sequence based on this RIL population (Evans, et al., 2013; Morishige, et al., 2013). Digital genotyping identified 1,462 SNP markers segregating in the RIL population and data on recombination frequency was used to create a 1139 cM genetic map spanning the 10 sorghum chromosomes.

Flowering time QTL were mapped in this population by phenotyping the RIL population for days to half pollen shed in greenhouses in 14 h long days (LD), 10 h short days (SD), and under field conditions. The BTx642/Tx7000 RIL population (n=90) and parental lines were grown under field conditions in a replicated randomized block design near College Station, Tex., in three consecutive years with planting between April 1-14. Days to mid-anthesis (pollen shed) were determined as a measure of flowering time. In the field, day-lengths increased from ˜12.6 h in April to 14.3 h in July, with an average daily maximum temperature of 31.7° C. and an average daily minimum temperature of 20.0° C. Ten plants of each RIL and the parental lines were grown in a greenhouse in 10 h day lengths (SD) or 14 h day lengths (LD) and phenotyped for flowering time in a similar manner as the populations grown in the field. RIL105 and RIL112 correspond to 4_6 and 12_14 in the original BTx642/Tx7000 RIL population (Xu, et al., 2000).

Tx7000 flowered in 73 days and BTx642 flowered approximately 4 days later under field conditions in College Station, Tex. When grown in a greenhouse at constant 14 h day lengths (LD) during the summer, Tx7000 flowered in 84 days and BTx642 flowered approximately 19 days later. When Tx7000 and BTx642 were grown in a greenhouse under 10 h day lengths (SD) during the winter, Tx7000 flowered in 54 days whereas BTx642 flowered approximately 11 days later.

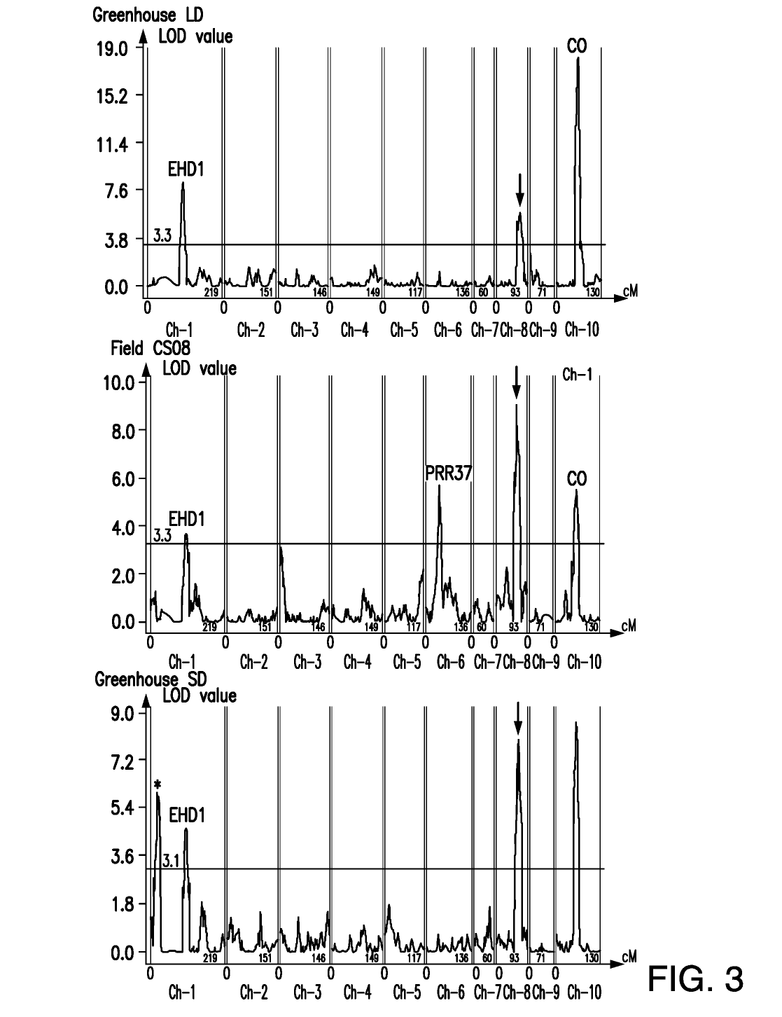

WinQTL Cartographer was used to identify flowering time QTL using flowering time data collected from each location/growing condition (FIG. 3). Genotyping by sequencing was carried out using Digital Genotyping (DG) (Morishige, et al., 2013) on the 90 RILs derived from BTx642 and Tx7000 (Evans, et al., 2013). A genetic linkage map was constructed using data generated from 1462 polymorphic DG markers using Mapmaker/EXP ver. 3.0b where recombination frequency was calculated using the Kosambi mapping function. QTLs were detected using Composite Interval Mapping (CIM) in WinQTL Cartographer v2.5 (Wang, et al., 2012). Significant LOD thresholds for QTL detection were determined based on experiment-specific permutations with 1000 repeats at α=0.05 (Churchill, et al., 1994). In QTL-based epistasis analysis, the 90 RILs were categorized into subpopulations based on alleles of SbPRR37 or alleles of SbCO respectively. Sub-populations homozygous for each allele of SbPRR37 and each allele of SbCO were then subjected to QTL analysis.

Three QTL for flowering time were observed in every environment and two additional QTL were identified in only one environment (Table 1).

TABLE 1
Parameters of flowering time QTL in BTx642/Tx7000 RIL population.

| | | | | | | | |
|---|---|---|---|---|---|---|---|
| QTL | Candidate gene | Chromosome number | Position (cM)^{a} | LOD score | Peak coordinate^{b} | Additive effect^{c} | R^{2d} |

Greenhouse LD (14 h)

| | | | | | | | |
|---|---|---|---|---|---|---|---|
| 1 | EHD1 | Chr_01 | 102.7 | 8.31 | 22012456-22012527 | −6.25 | 0.12 |
| 2 | ND^{e} | Chr_08 | 67.9 | 5.82 | 50255989-50256060 | −5.02 | 0.08 |
| 3 | CO | Chr_10 | 61.7 | 18.43 | 13696999-13697070 | −12.69 | 0.40 |

Field LD condition CS08

| | | | | | | | |
|---|---|---|---|---|---|---|---|
| 1 | EHD1 | Chr_01 | 102.7 | 3.74 | 22012456-22012527 | −1.09 | 0.09 |
| 2 | PRR37 | Chr_06 | 42.0 | 5.71 | 40201054-40201125 | 1.53 | 0.15 |
| 3 | ND | Chr_08 | 60.2 | 9.09 | 49290307-49290378 | −1.80 | 0.26 |
| 4 | CO | Chr_10 | 59.7 | 4.11 | 10080053-10080126 | −1.50 | 0.16 |

Greenhouse SD (10 h)

| | | | | | | | |
|---|---|---|---|---|---|---|---|
| 1 | ND | Chr_01 | 16.3 | 6.00 | 7208344-7208415 | 2.18 | 0.09 |
| 2 | EHD1 | Chr_01 | 102.7 | 4.92 | 22012456-22012527 | −1.80 | 0.07 |
| 3 | ND | Chr_08 | 65.1 | 7.96 | 49797259-49797330 | −2.46 | 0.14 |
| 4 | CO | Chr_10 | 59.7 | 8.70 | 10080053-10080126 | −3.30 | 0.17 |

^{a}Position of likelihood peak (highest LOD score).
^{b}Peak coordinate: physical coordinate of the likelihood peak.
^{c}Additive effect: A positive value means the delay of flowering time due to Tx7000 allele. A negative value means the delay of flowering time due to BTx642 allele.
^{d}R² (coefficient of determination): percentage of phenotypic variance explained by the QTL.
^{e}ND: Candidate gene is not determined.

### Example 2

### QTL on Chromosome 1 Including Candidate Gene SbEHD1

A flowering time QTL discovered on chromosome 1 (SBI-01; 19.2-22.0 Mbp) explained 12.3% of the phenotypic variance for flowering time in a LD greenhouse environment. SbEHD1, located on SBI-01 was found in a one LOD interval spanning this QTL. (Sorghum bicolor EHD1; Locus Name: Sobic.001G227900; Alias: Sb01g019980, Location: Chromosome 01; Gene Coordinates (BTx623; Phytozome v2.1): Chr01:21816924..21823874 forward).

There were no amino acid differences between the SbEhd1 protein sequences in Tx7000 and BTx623. However, comparison of SbEhd1 protein sequences from BTx642 and Tx7000 revealed two amino acid substitutions, Asp144Asn and Thr157Ile. The differences in SbEhd1 protein sequences were found in a GARP domain that is highly conserved among OsEHD1, SbEHD1, and ARABIDOPSIS RESPONSE REGULATOR 1/2 (ARR1/2). The SbEHD1 allele identified in BTx642 within the flowering time QTL discovered on SBI-01 (designated SbEhd1-2) delays flowering in LD and SD relative to the SbEHD1 allele in Tx7000 (designated SbEHD1-1).

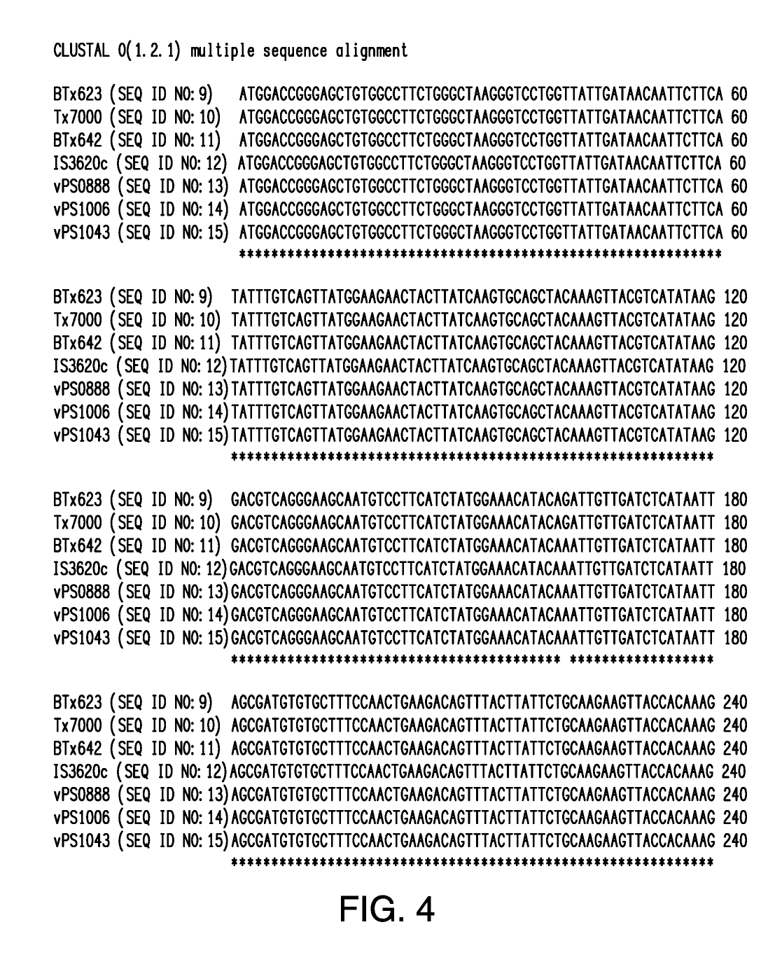

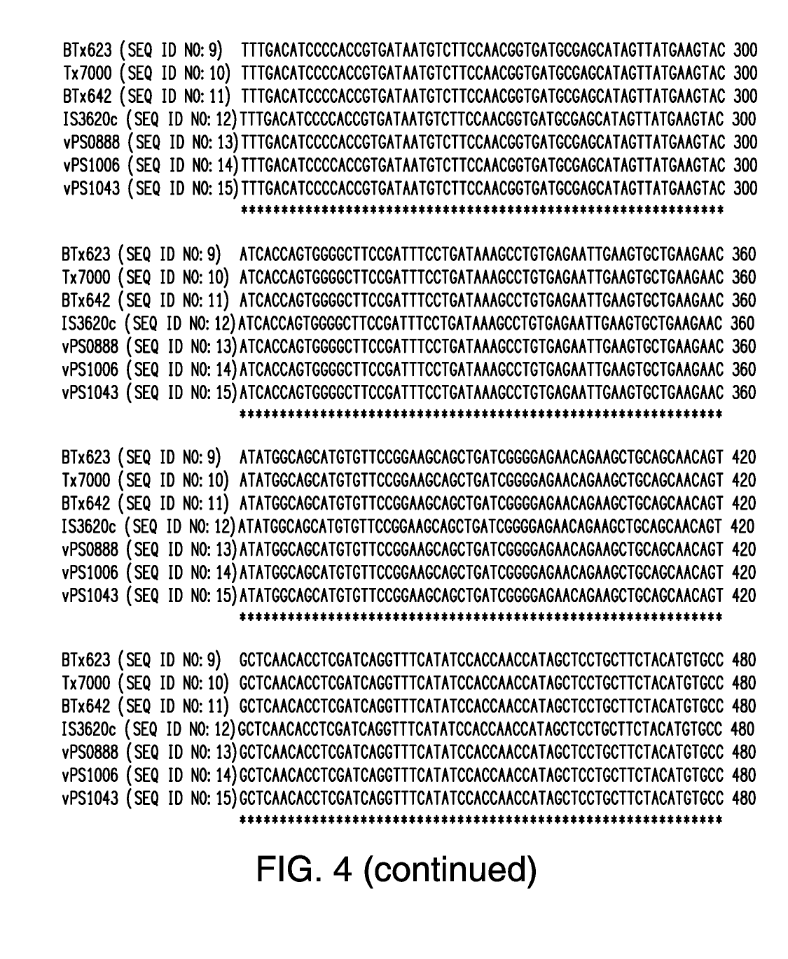

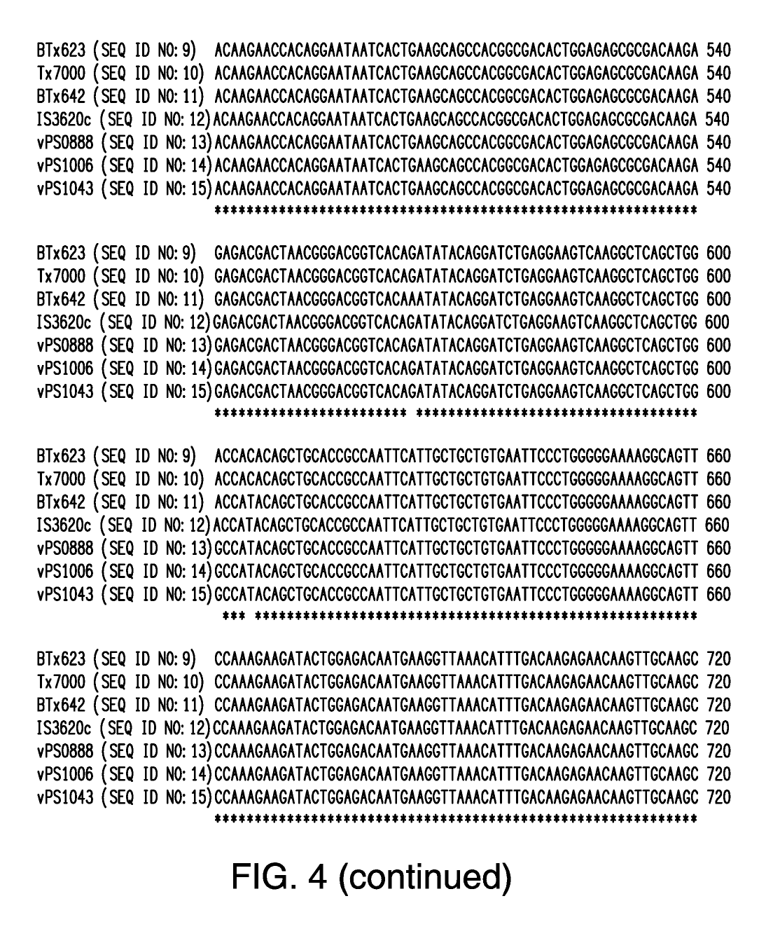

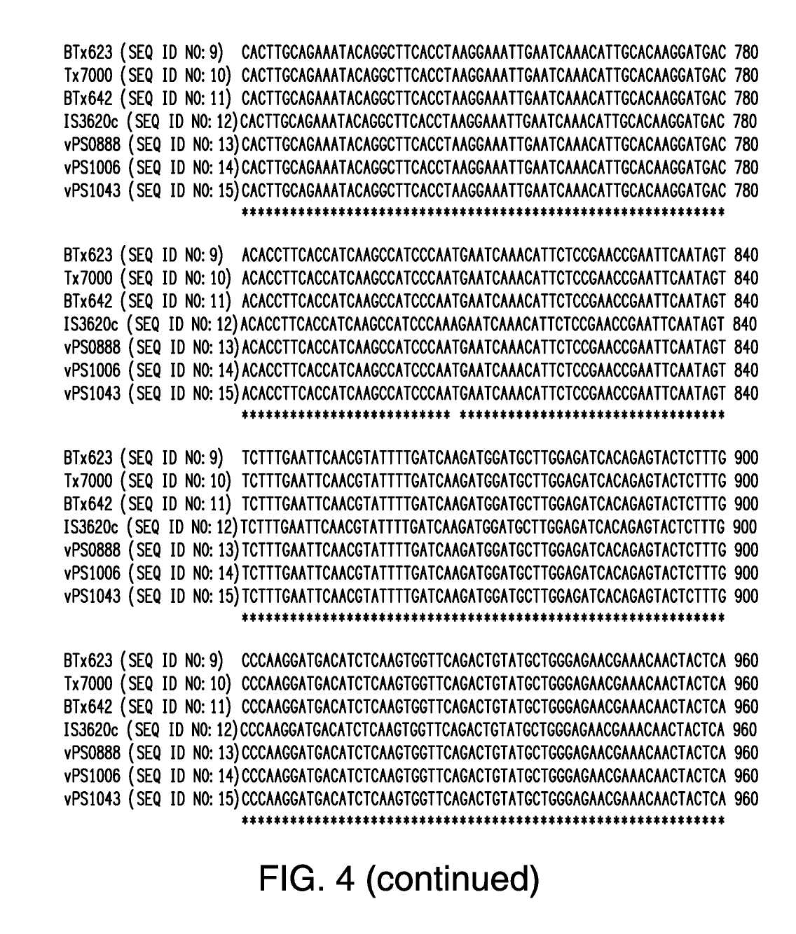

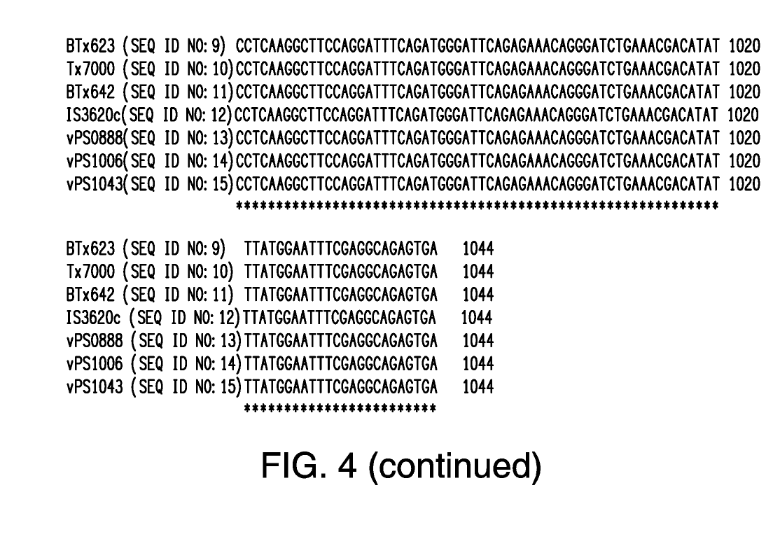

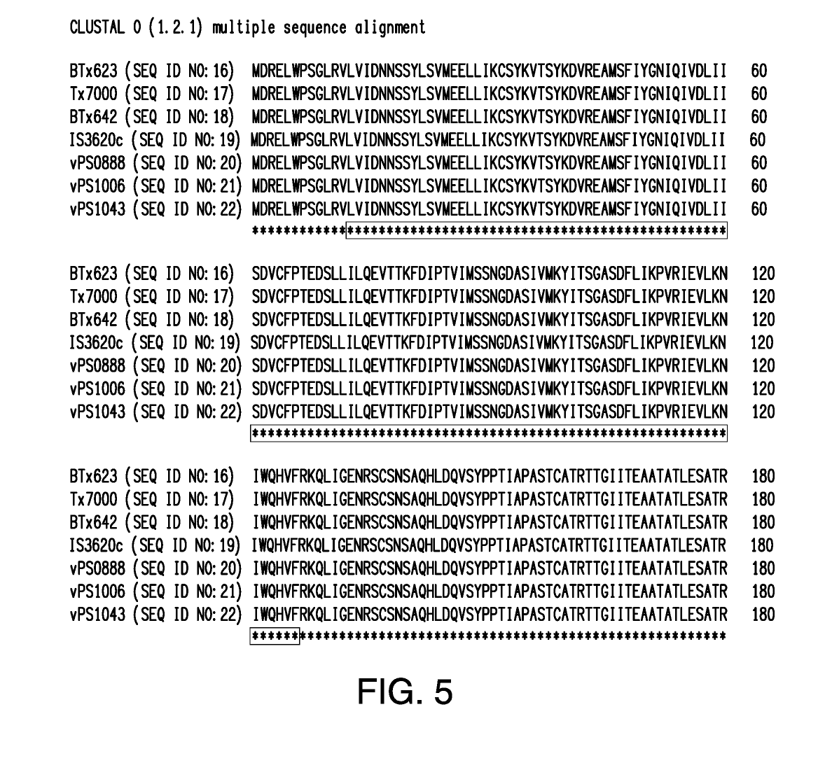

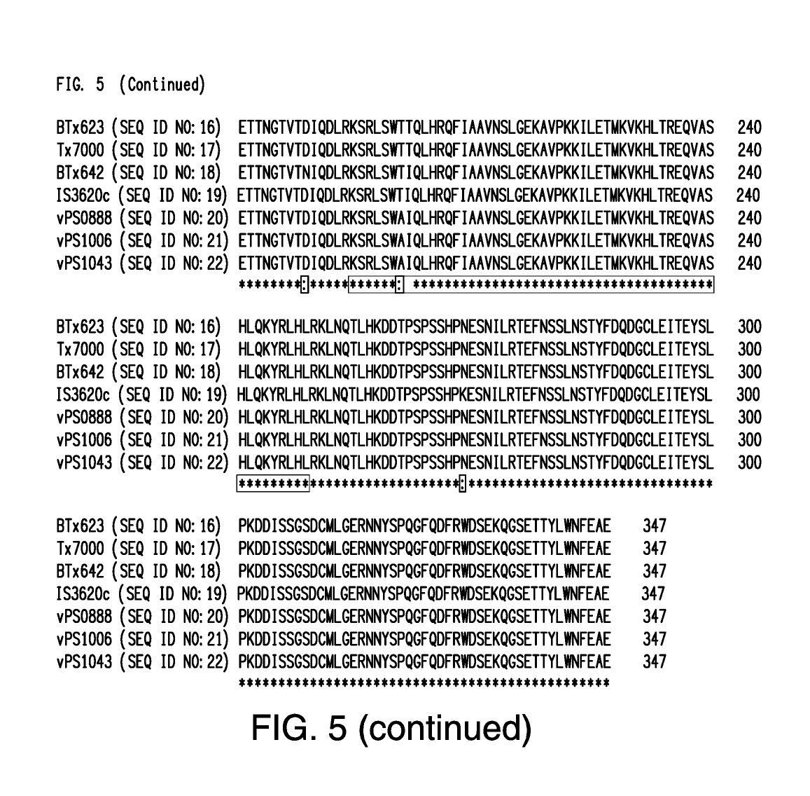

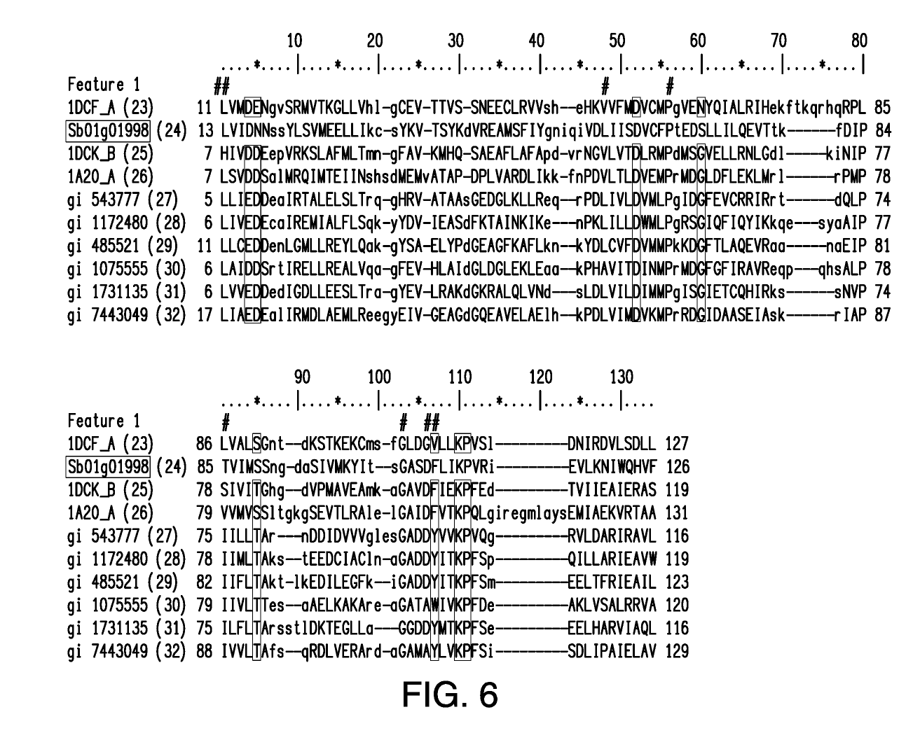

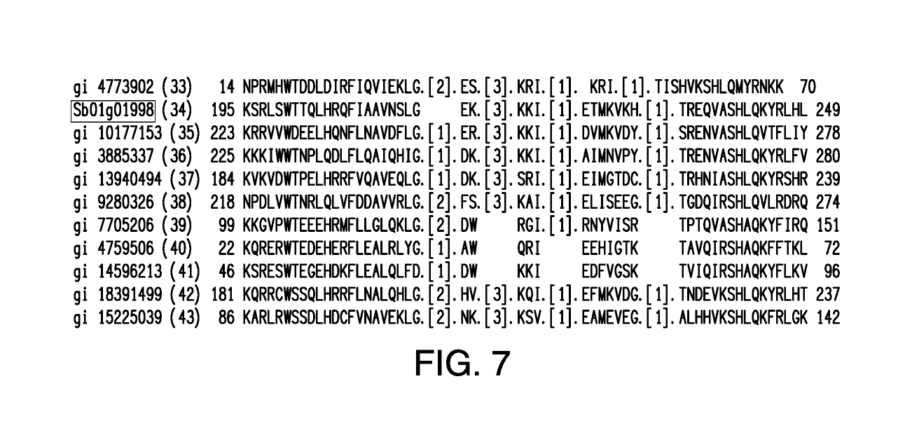

In order to further investigate the newly identified flowering time QTL on SBI-01, the region surrounding SbEHD1 was sequenced from several lines, as shown in Table 2. Sequence alignments of SbEHD1 mRNA sequences (FIG. 4) and protein sequences (FIG. 5) from several lines were made. FIG. 6 shows an alignment of a conserved signal receiver domain within the Ehd1 protein sequence, and FIG. 7 shows an alignment of a conserved DNA-binding domain within the Ehd1 protein sequence.

TABLE 2
Sequenced region surrounding SbEHD1 (Sb01g019980).

| | Total Genomic | mRNA (bases; | Protein |
|---|---|---|---|
| | Region Sequenced | coding region | (amino acid |
| Genotype | (bp) | only) | residues) |
| BTx623 | 9611 | 1044 | 347 |
| BTx642 | 9611 | 1044 | 347 |
| Tx7000 | 9611 | 1044 | 347 |
| IS3620c | 9469 | 1044 | 347 |
| vPS0888 | 9635 | 1044 | 347 |
| vPS1006 | 9641 | 1044 | 347 |
| vPS1043 | 9637 | 1044 | 347 |

Mutations were identified in the mRNA coding region corresponding to SbEHD1, as shown in Table 3.

TABLE 3
Mutations identified in the EHD1 mRNA and corresponding amino acid changes.

| | Mutation in mRNA | | | | |
|---|---|---|---|---|---|
| | Chr01:21819496 | Chr01:21822559 | Chr01:21822595 | Chr01:21822599 | Chr01:21823314 |
| Amino Acid | (G > A) | (G > A) | (A > G) | (C > T) | (T > A) |
| Residue Change | none | D189N | T201A | T202I | N269K |
| BTx623 | − | − | − | − | − |
| BTx642 | − | + | − | + | − |
| Tx7000 | − | − | − | + | − |
| IS3620c | − | − | − | + | + |
| VPS0888 | − | − | + | + | − |
| vPS1006 | − | − | + | + | − |
| vPS1043 | − | − | + | + | − |

The recessive alleles of SbEHD1 identified in BTx642 (Sbehd1-1), IS3620C (Sbedh1-2), and vPS 1043 (Sbehd1-3) delay flowering time in short days and long days.

### Example 3

### QTL on Chromosome 10 Including Candidate Gene SbCO

A flowering time QTL located on SBI-10 (10.1-13.7 Mbp) was observed in all environments. This QTL spans a region that encodes a homolog of CONSTANS (CO) (Sorghum bicolor CONSTANS; Locus Name: Sobic.010G115800; Alias: Sb10g010050; Location: Chromosome 10; Gene Coordinates (BTx623; Phytozome v2.1): Chr10:12284504..12286660 forward) and HEADING DATE1 (Hd1). The QTL spanning the sorghum homolog of CONSTANS explained ˜40% of the variance in flowering time in LD greenhouses, and 16-17% when plants were grown in the field or SD greenhouses (Table 1).

Several lines were sequenced, and mutations were identified in the mRNA coding region corresponding to CO, as shown in Table 4. A unique low or null activity allele of SbCO (designated Sbco-3) was identified in BTx642. The Sbco-3 allele has a His106Tyr substitution that within a B-box2 domain. RILs identical at other flowering loci but that contain the Sbco-3 allele flower 10-14 days later in short days and up to 30 days later in long days compared to plants with fully active SbCO-1.

TABLE 4
Mutations identified in SbCO mRNA and
corresponding amino acid changes.

| | Mutation in mRNA |
|---|---|
| | Chr10:12285108 |
| Amino Acid | (C > T) |
| Residue Change | H106Y |
| SbCO-1 (BTx623) | − |
| SbCO-2 (Tx7000) | − |
| Sbco-3 (BTx642) | + |

### Example 4

### Identification of a CONSTANS Homolog in Sorghum

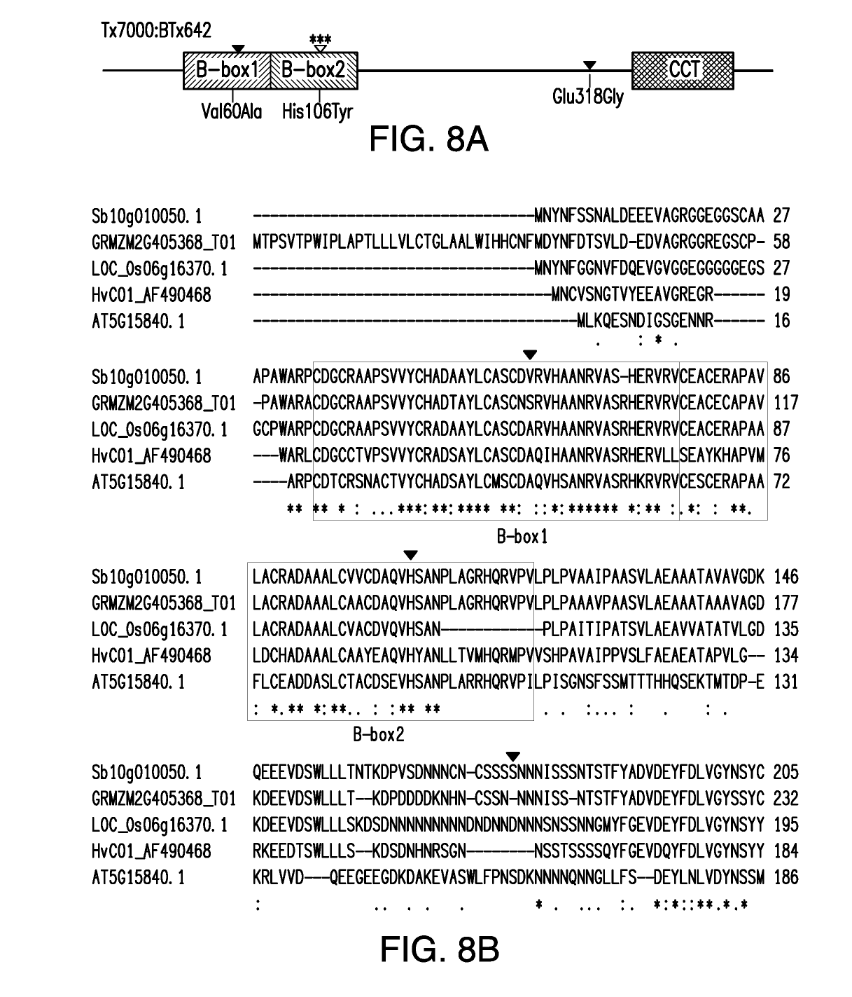

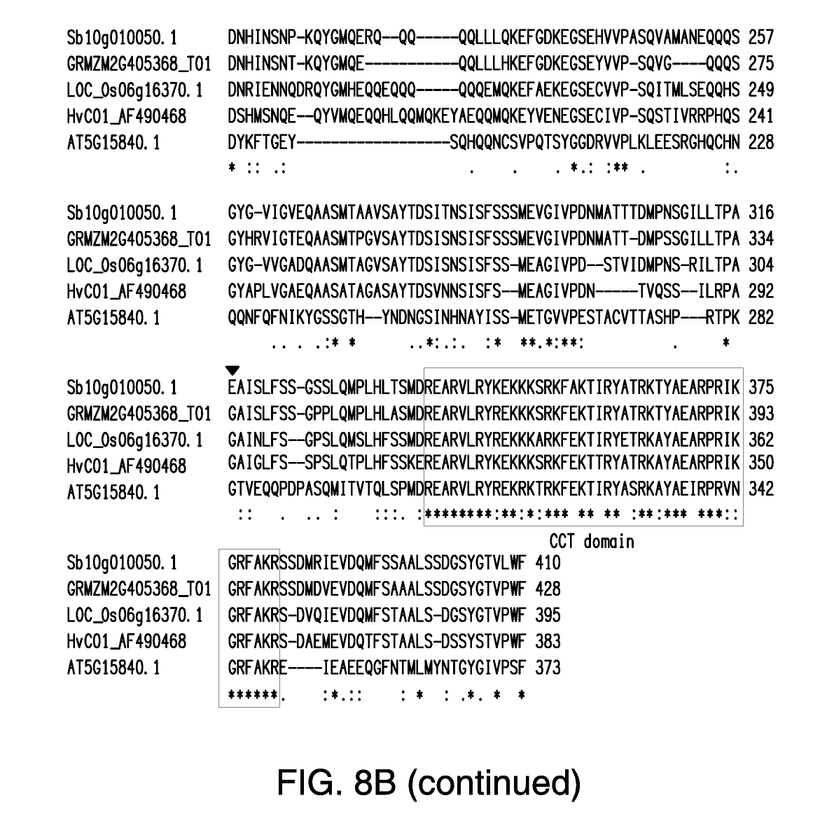

The hypothesis that the flowering time QTL on SBI-10 was caused by alleles of a candidate CONSTANS/Hd1 homolog was investigated further through gene sequence alignment and analysis of colinearity. The amino acid sequence of rice Hd1 was used to identify homologs in sorghum, maize, barley and Arabidopsis using data from Phytozome v9.1 (http://www.phytozome.net/). Sb10g010050 (score=71.9), GRMZM2G405368_T01 (score=80.7), AF490468 (score=63.2) and AT5G15850 (score=40.5) had the highest similarity to Hd1 in each species. GRMZM2G405368_T01 and AF490468 were previously identified as the maize CONSTANS-like gene, conz1 (Miller, et al., 2008) and barley CONSTANS-like gene, HvCO1 (Campoli, et al., 2012), respectively, while AT5G15850 encodes CO in Arabidopsis (Robson, et al., 2001). Multiple sequence alignment of the CO homologs showed that Sb10g010050 has all of the characteristic protein domains found in CONSTANS-like gene families (FIG. 8), including an N-terminal B-box1 (residues 35-76), B-box2 (residues 77-120) domains and a C-terminal CCT domain (residues 339-381). The candidate sorghum homolog of CONSTANS (Sb10g010050) is located on SBI-10 and rice Hd1 (Os06g16370) is located on the homologous rice chromosome 6, suggesting that these genes may be orthologs. The sequences of these genes and adjacent sequences in each chromosome were aligned to determine if SbCO and OsHd1 were in a region of gene colinearity. The sorghum sequences flanking Sb10g010050 were downloaded from Phytozome and aligned with sequences from rice chromosome 6 flanking Hd1 using GEvo (Genome Evolution Analysis; http://genomevolution.org/CoGe/GEvo.p1). Three genes and Hd1 were aligned and in the same relative order in a 100 kbp region in the two chromosomes, consistent with the identification of Sb10g010050 as an ortholog of rice Hd1. Therefore, based on sequence similarity and colinearity, Sb10g010050 was designated as an ortholog of rice Hd1 and a probable ortholog of Arabidopsis CO and termed “SbCO.”

The hypothesis that the flowering time QTL on SBI-10 was associated at least in part with different alleles of the newly identified SbCO gene in BTx642 and Tx7000 was investigated further by comparing the SbCO sequences from these genotypes. The comparison revealed one difference in intron sequence and four differences in the coding region, three of which cause changes in amino acid sequence (Table 5). The amino acid change Va160Ala, occurs in B-box1 (FIG. 8, black arrow), and represents a conservative change in amino acid sequence that is expected to be tolerated based on SIFT analysis (Kumar, et al., 2009). The amino acid change Glu318Gly occurs outside the B-boxes and CCT-domain (FIG. 8, black arrow) and was also predicted to be tolerated based on SIFT analysis. While the Val60Ala and Glu318Gly changes in protein sequence may not disrupt CO function, it is possible that other aspects of CO could be modified by these differences. The His106Tyr change in BTx642 CO protein sequence located in B-box2 (FIG. 8) is predicted to disrupt CO function. In the wild type version of CONSTANS, His106 is required for zinc coordination and protein activity (Valverde, et al., 2011). The BTx642 allele of CONSTANS was designated Sbco-3 because the Arabidopsis allele co-3 has the same His106Try substitution that disrupts function. The wild type alleles of CO in BTx623 and Tx7000 had identical CO protein sequences except for a Ser177Asn substitution in Tx7000 (FIG. 8B), a modification that does not affect the B-boxes or the CCT domain, and is predicted by SIFT to have minimal impact on CO function. Based on this analysis, the CONSTANS alleles in BTx623 and Tx7000 were designated as SbCO-1 and SbCO-2, respectively, and the allele in BTx642 as Sbco-3. BTx642 (Sbco-3) flowers later than Tx7000 (SbCO-2) in both long and short days.

TABLE 5
Characterization of SbCO alleles from BTx623, Tx7000, and BTx642.

| | SNP # | | | | | |
|---|---|---|---|---|---|---|
| | 1 | 2 | 3 | 4 | 5 | 6 |
| Location (SBI-10) | 12275306 | 12275331 | 12275443 | 12275657 | 12276109 | 12276334 |
| Nucleotide variation | T > C | T > G | C > T | G > A | C > T | A > G |
| Protein modification | Val60Ala | No change | His106Tyr | Ser177Asn | intron | Glu318Gly |
| CONSTANS domain | β-box1 | | β-box2 | | | |
| SIFT score | tolerant | N/A* | Intolerant | Tolerant | N/A | Tolerant |
| sbCO-1 (BTx623) | − | − | − | − | − | − |
| sbCO-2 (Tx7000) | − | − | − | + | − | − |
| sbco-3 (BTx642) | + | + | + | + | + | + |

*N/A: Not applicable

### Example 5

### SbCO Alleles Modulate Expression of Genes in the Flowering Time Pathway

The influence of SbCO alleles on the expression of other genes in the flowering-time regulatory pathway was analyzed to further understand how SbCO affects flowering time. RIL105 and RIL112 were identified that differ in alleles of SbCO but not at the other main loci that affect flowering time. RIL105 and RIL112 are homozygous for BTx642 alleles for the flowering time QTL on SBI-01 (spanning Sbehd1-2), SBI-06 (spanning Sbprr37-1), and SBI-08. BTx642 encodes a null allele of Ma1 (Sbprr37-1), a gene that contributes to photoperiod sensitivity. Tx7000 contains a weak allele of Ma1 (Sbprr37-2) that encodes a full-length protein that inhibits flowering based on QTL analysis. Therefore, RIL105 and RIL112 were selected for expression studies because both contain DNA from BTx642 on SBI-06 from 0-42 Mbp, ensuring that these genotypes are null for Ma1 (Sbprr37-1). In addition, both RILs encode a null allele of Ma6 (Sbghd7-1) located at the proximal end of SBI-06. Therefore, comparison of gene expression in RIL105 and RIL112 caused by differences in SbCO alleles will not be influenced by Ma1 or Ma6 the main determinants of photoperiod sensitivity in sorghum.

When grown in a LD greenhouse, RIL105 (SbCO-2) flowered in ˜75 days, whereas RIL112 (Sbco-3) flowered in ˜113 days consistent with the hypothesis that SbCO functions as an activator of flowering. When grown in a SD greenhouse, RIL105 (SbCO-2) flowered in ˜55 days, whereas RIL112 (Sbco-3) flowered in ˜72 days, consistent with the hypothesis that SbCO functions as an activator of flowering in short days in sorghum.

### Example 6

### Additional Novel QTL Associated with Flowering Time

A flowering time QTL located on SBI-08 (48.1-50.3 Mbp) was observed in LD, SD, and under field conditions (FIG. 3). This QTL explained 8-14% of the phenotypic variance in LD and SD and 18-22% of the variance in field environments.

A flowering time QTL located at the end of SBI-01 (˜7.2 Mbp) was observed only when the BTx642/Tx7000 RIL population was grown in the SD greenhouse (FIG. 3C).

### Example 7

### Additional Alleles Associated with Flowering Time

### SbCN8, SbCN12, SbCDF1, SbEHD3, and SbELF3

Allelic variants of SbCN8, SbCN12, SbCDF1, SbEHD3, and SbELF3 have been identified in various genotypes. Allelic variants of SbCN8, SbCN12, SbCDF1, SbEHD3, and SbELF3 are used in conjunction with favorable alleles of SbEHD1 and SbCO to produce plants with delayed flowering in short and long day environments. Allelic variants of SbCN8, SbCN12, SbCDF1, SbEHD3, and SbELF3 are added to first or subsequent generation R-lines and A/B-lines containing recessive alleles of Sbehd1-3 and or Sbco-3 to further delay flowering short and long day environments.

### SbPRR37, SbGHD7 and SbPHYC

Allelic variants of SbPRR37, SbGHD7 and SbPHYC have been identified in various genotypes, and are useful for construction of hybrids that show delayed flowering in long days due to photoperiod sensitivity. Alleles of SbPRR37, SbGHD7, and SbPHYC are used in conjunction with favorable alleles of SbEHD1 and SbCO to create R-lines and B-lines that exhibit delayed flowering in short days and that are also photoperiod sensitive with delayed flowering in long days.

### Example 8

### Development of Sorghum Lines Exhibiting Delayed Flowering

Weak or null alleles of SbCO or SbEHD1 are deployed in R-lines and A/B-lines used for hybrid seed production and hybrid plant production. This reduces the level and activity of these activators of SbCN8 and SbCN12, resulting in delayed flowering in the hybrids in short and long days. This technology is also used in conjunction with the Ma1/Ma5/Ma6 loci that delay flowering in long days to optimize flowering time in all production locations.

### Example 9

### Production of Plants Comprising Sbco-3 Alleles

BTx642 plants comprising low or null activity alleles of Sbco-3 are crossed to B-lines and R-lines useful for production of sweet sorghum or energy sorghum hybrids to produce plants which flower later in short days and long days compared with plants comprising fully active SbCO. Progeny plants homozygous recessive for Sbco-3 are selected. Progeny plants may be selected using genetic markers within or genetically linked to the genomic segment spanning 10.1-13.7 Mbp on chromosome 10, or within or genetically linked to the SbCO gene.

### Example 10

### Production of Plants Comprising Sbehd1-1, Sbehd1-2, or Sbehd1-3 Alleles

Plants comprising the low or null activity alleles Sbehd1-1, Sbehd1-2, or Sbehd1-3 are crossed to B-lines and R-lines useful for production of sweet sorghum or energy sorghum hybrids to produce plants which with delayed flowering compared with plants comprising fully active SbEHD1. Progeny plants homozygous recessive for Sbehd1-1, Sbehd1-2, or Sbehd1-3 are selected. Progeny plants may be selected using genetic markers within or genetically linked to a genomic segment spanning 19.2-22.0 Mbp on chromosome 1, or within or genetically linked to the SbEHD1 gene.

### Example 11

### Production of Plants Comprising Combinations of Sbco and Sbehd1 Alleles

Plants comprising low or null activity alleles of Sbehd1-1, Sbehd1-2, or Sbehd1-3 are crossed to B-lines and R-lines comprising low or null activity alleles of Sbco-3 useful for production of sweet sorghum or energy sorghum hybrids. Progeny plants exhibit delayed flowering compared with plants comprising fully active SbEHD1, or SbCO.

R-lines and A/B lines are developed from this material using MAB or genome selection for lines homozygous for a combination of SbEHD1 recessive alleles (i.e., Sbehd1-1, Sbehd1-2 or Sbehd1-3) and Sbco-3. Lines only homozygous for Sbehd1-3 will also be useful to select and use for production of hybrids.

A/B-lines comprising various combinations of recessive SbEHD1 and SbCO alleles are crossed R-lines comprising various combinations of recessive SbEHD1 and SbCO alleles and produce hybrid seed.

A/B-lines homozygous for Sbehd1-3 and Sbco-3 are crossed with R-lines homozygous for Sbehd1-3 and Sbco-3 to produce hybrids with delayed flowering times compared with plants comprising fully active SbEHD1 and SbCO.

Hybrids comprising various combinations of recessive SbEHD1 and SbCO alleles are grown in short day and long day environments, and hybrids with specific days to flowering are identified which are optimal for production in specific growing regions or for specific purposes.

### Example 12

### Production of Plants Comprising Combinations of Sbco, Sbehd1, and Other Alleles

Plants comprising recessive Sbco-3 and Sbehd1 alleles that delay flowering in short days are crossed with plants comprising alleles of Ma1, Ma5, and Ma6 that delay flowering in long days (R-lines=Ma1, Ma6, ma5; A/B-lines=ma1, ma6, Ma5).

Flowering times of some plant lines are further refined for optimal production by introducing modifiers of SbCO (i.e., alleles of CDF1) or modifiers of SbEHD1 (i.e., alleles of SbEHD2, SbELF3).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

Campoli C, Drosse B, Searle I, Coupland G, von Korff M: Functional characterization of HvCO1, the barley (Hordeum vulgare) flowering time ortholog of CONSTANS. Plant J 2012, 69(5):868-880.

Churchill G A, Doerge R W: Empirical threshold values for quantitative trait mapping. Genetics 1994, 138(3):963-971.

Evans J, McCormick R F, Morishige D, Olson S N, Weers B, Hilley J, Klein P, Rooney W, Mullet J: Extensive variation in the density and distribution of DNA polymorphism in sorghum genomes. PLoS One 2013, 8(11):e79192.

Kumar P, Henikoff S, N g P C: Predicting the effects of coding non-synonymous variants on protein function using the SIFT algorithm. Nat Protoc 2009, 4(7):1073-1081.

Miller TaA, Muslin E H, Dorweiler J E: A maize CONSTANS-like gene, conz1, exhibits distinct diurnal expression patterns in varied photoperiods. Planta 2008, 227(6):1377-1388.

Morishige D T, Klein P E, Hilley J L, Sahraeian S M, Sharma A, Mullet J E: Digital genotyping of sorghum—a diverse plant species with a large repeat-rich genome. BMC Genomics 2013, 14(1):448.

Robson F, Costa M M, Hepworth S R, Vizir I, Pineiro M, Reeves P H, Putterill J, Coupland G: Functional importance of conserved domains in the floweringtime gene CONSTANS demonstrated by analysis of mutant alleles and transgenic plants. Plant J 2001, 28(6):619-631.

Valverde F: CONSTANS and the evolutionary origin of photoperiodic timing of flowering. J Exp Bot 2011, 62(8):2453-2463.

Wang S, Basten C J, Zeng Z-B: Windows QTL Cartographer 2.5. In Department of Statistics. Raleigh, N.C.: North Carolina State University; 2012.

Xu W, Subudhi P K, Crasta O R, Rosenow D T, Mullet J E, Nguyen H T: Molecular mapping of QTLs conferring stay-green in grain sorghum (Sorghum bicolor L. Moench). Genome 2000, 43(3):461-469.

Yang S, Weers B D, Morishige D T, Mullet J: CONSTANS is a photoperiod regulated activator of flowering in sorghum. BMC Plant Biology 2014, 14:148.

1. A method of obtaining a sorghum plant exhibiting delayed or early flowering time comprising:
a) providing a population of sorghum plants;
b) detecting in said population a plant comprising a delayed flowering time allele at a polymorphic locus in, or genetically linked to, a chromosomal segment between approximately 19.2 Mbp and 22.0 Mbp on chromosome 1; or at a polymorphic locus in, or genetically linked to, a chromosomal segment between approximately 6.2 Mbp and 8.2 Mbp on chromosome 1; or at a polymorphic locus in, or genetically linked to, a chromosomal segment between approximately 48.1 Mbp and 50.3 Mbp on chromosome 8; or at a polymorphic locus in, or genetically linked to, a chromosomal segment between approximately 10.1 Mbp and 13.7 Mbp on chromosome 10; and
c) selecting said plant from said population based on the presence of said allele;
wherein said plant exhibits delayed or early flowering compared to a control plant lacking said delayed flowering time allele.

2. The method of claim 1, wherein said polymorphic locus is in or genetically linked to SbEHD1 or SbCO.

3. The method of claim 2, wherein said SbEHD1 gene encodes an Sbehd1 protein comprising a mutation at a position homologous to amino acid 189, 201, 202, or 269 of SEQ ID NO: 4 relative to SEQ ID NO: 4.

4. The method of claim 2, wherein said SbCO gene encodes an SbCO protein comprising a mutation at a position homologous to amino acid 106 of SEQ ID NO: 8 relative to SEQ ID NO: 8.

5. The method of claim 1, wherein step (a) of providing comprises crossing a first sorghum plant comprising a delayed flowering time allele with a second sorghum plant to produce a population of sorghum plants.

6. The method of claim 5, wherein producing said population of sorghum plants comprises selfing or backcrossing.

7. The method of claim 1, wherein step (b) of detecting comprises the use of an oligonucleotide probe.

8. A method of producing a sorghum plant exhibiting delayed or early flowering time comprising:
a) crossing a first sorghum plant comprising a delayed flowering time allele with a second sorghum plant of a different genotype to produce one or more progeny plants; and
b) selecting a progeny plant based on the presence of said allele at a polymorphic locus in, or genetically linked to, a chromosomal segment between approximately 19.2 Mbp and 22.0 Mbp on chromosome 1; or at a polymorphic locus in, or genetically linked to, a chromosomal segment between approximately 6.2 Mbp and 8.2 Mbp on chromosome 1; or at a polymorphic locus in, or genetically linked to, a chromosomal segment between approximately 48.1 Mbp and 50.3 Mbp on chromosome 8; or at a polymorphic locus in, or genetically linked to, a chromosomal segment between approximately 10.1 Mbp and 13.7 Mbp on chromosome 10;
wherein said allele confers delayed or early flowering time compared to a plant lacking said allele.

9. The method of claim 8, wherein said polymorphic locus is in or genetically linked to SbEHD1 or SbCO.

10. The method of claim 9, wherein said SbEHD1 gene encodes an Sbehd1 protein comprising a mutation at a position homologous to amino acid 189, 201, 202, or 269 of SEQ ID NO: 4 relative to SEQ ID NO: 4.

11. The method of claim 9, wherein said SbCO gene encodes an SbCO protein comprising a mutation at a position homologous to amino acid 106 of SEQ ID NO: 8 relative to SEQ ID NO: 8.

12. The method of claim 8, wherein step b) of selecting further comprises selecting a progeny plant which is homozygous for said allele.

13. The method of claim 8, further comprising:
c) crossing said progeny plant with itself or a second plant to produce one or more further progeny plants; and
d) selecting a further progeny plant comprising said allele.

14. The method of claim 13, wherein step (d) of selecting comprises marker-assisted selection.

15. The method of claim 13, wherein said further progeny plant is an F2-F7 progeny plant.

16. The method of claim 13, wherein producing the progeny plant comprises selfing or backcrossing.

17. The method of claim 16, wherein backcrossing comprises from 2-7 generations of selfing or backcrossing.

18. The method of claim 16, wherein selfing or backcrossing comprises marker-assisted selection.

19. The method of claim 18, wherein selfing or backcrossing comprises marker-assisted selection in at least two generations.

20. The method of claim 19, wherein selfing or backcrossing comprises marker-assisted selection in all generations.

21. The method of claim 8, wherein said first sorghum plant is an inbred or a hybrid.

22. The method of claim 8, wherein said second sorghum plant is an agronomically elite sorghum plant.

23. The method of claim 22, wherein said agronomically elite sorghum plant is an inbred or a hybrid.

24. The method of claim 23, wherein said agronomically elite sorghum plant is from sorghum line BTx642.

25. A sorghum plant produced by the method of claim 1.

26. A plant part of the sorghum plant of claim 25.

27. A seed that produces the sorghum plant of claim 25.

28. A method of producing biofuel, comprising the steps of:
(Original) harvesting biomass from the sorghum plant of claim 25; and
(Original) producing biofuel from said biomass.

29. The use of the plant part of claim 26 in the production of biofuels.

30. A sorghum plant produced by the method of claim 8.
